(19) United States
(12) Patent Application Publication
(10) Pub. No.: US 2016/0250208 A1
(43) Pub. Date: Sep. 1, 2016

(54) TREATING ORGAN-SPECIFIC T CELL MEDIATED AUTOIMMUNE DISEASES

(71) Applicant: UNIVERSITY OF MASSACHUSETTS, Boston, MA (US)

(72) Inventors: Joonsoo Kang, Holden, MA (US); Leslie Berg, Shrewsbury, MA (US); Nitya Jain, Worcester, MA (US)

(21) Appl. No.: 15/027,399

(22) PCT Filed: Oct. 10, 2014
(86) PCT No.: PCT/US2014/060096

§ 371 Date: Apr. 5, 2016

Related U.S. Application Data
(60) Provisional application No. 61/889,649, filed on Oct. 11, 2013.

(51) Int. Cl. A61K 31/496; A61K 45/06

(52) U.S. Cl. CPC A61K 31/496; A61K 45/06

(57) ABSTRACT

The specification provides methods of treating a subject with an organ-specific T cell mediated autoimmune disease.

### CROSS-REFERENCE TO RELATED APPLICATIONS

This application claims the benefit of U.S. Patent Application No. 61/889,649, filed Oct. 11, 2013. The contents of the foregoing are incorporated herein by reference in their entirety.

### FEDERALLY SPONSORED RESEARCH OR DEVELOPMENT

This invention was made with Government support under Grant Nos. RC1 DK086474, AI083505, and AI054670, awarded by the National Institutes of Health. The Government has certain rights in the invention.

### TECHNICAL FIELD

Described herein are methods for the treatment of organ-specific T cell mediated autoimmune diseases, e.g., Type 1 diabetes, Hashimoto's thyroiditis, multiple sclerosis, non-infectious uveitis, Sjögren's syndrome, primary biliary cirrhosis, autoimmune hepatitis, and ankylosing spondylitis, particularly where the method inhibits migration of autoreactive T cells into a target tissue.

### BACKGROUND

T lymphocyte-mediated organ specific autoimmune diseases such as Type 1 diabetes arise from the aberrant activation of self tissue antigen-reactive T cells, their migration to target tissues and destruction of the self antigen expressing cells, ultimately leading to organ failures. Immunotherapies for organ specific autoimmunity including corticosteroid regimens, blockade of inflammatory effectors and tolerance induction by altering T cell activation processes fail to target self-reactive T cells specifically, resulting in compromised general immunity and opportunistic infections that are sometimes fatal. A recent focused approach aimed at regulating self-reactive T cell migration to organs to limit immune pathology suffers from the same problem of dangerous pan-immune suppression during the therapy. The signaling pathways that can selectively modulate self-antigen specific T cells versus pathogen-specific T cells are unknown, and this knowledge gap has impeded the generation of drugs that only target pathogenic T cells. The present disclosure demonstrates that the CD28 costimulatory pathway of T cell activation is necessary for the trafficking of self-reactive, pathogenic T cells. The Tec-kinase, interleukin-2-inducible T cell kinase (ITK), is identified as a critical downstream mediator of the CD28 signal controlling autoreactive T cell migration. In contrast, ITK is dispensable for activation of naïve T cells per se to self and foreign antigens. Using the Ctla4-/- mouse as a model of T cell-mediated multi-organ autoimmunity, the present disclosure demonstrates that genetic and pharmacological inhibition of ITK prevents the migration of activated T cells into target tissues. Further, small molecule ITK inhibitors prevent pancreatic islet infiltration by diabetogenic T cells in mouse models of Type 1 diabetes. Critically, the loss of ITK function does not materially impair immunity to pathogens, identifying ITK as a central discriminator of self versus foreign T cell reactivity. These results show that pharmacological disruption of ITK function can be an effective treatment of human T cell mediated organ-specific autoimmune diseases, specifically targeting self-reactive T cells, while permitting pathogen sensing T cells to operate normally during a treatment window.

### SUMMARY

The present disclosure is based, in part, on the discovery that ITK and CD28 signals regulate autoreactive T cell trafficking and that organ-specific T cell mediated autoimmune diseases, e.g., Type 1 diabetes, Hashimoto's thyroiditis, multiple sclerosis, non-infectious uveitis, Sjögren's syndrome, primary biliary cirrhosis, autoimmune hepatitis, and ankylosing spondylitis, can be treated by inhibiting ITK. Accordingly, the present specification provides methods of treating organ-specific T cell mediated autoimmune diseases by identifying a subject in need of treatment for an organ-specific T cell mediated autoimmune disease and inhibiting ITK by administering to the subject a therapeutically effective amount of an ITK inhibitor, e.g., BMS509744, ibrutinib, 10n, 2-amino-5-(thioaryl)thiazole, 5 aminomethylbenzimidazole, 2-amino-5-[(thiomethyparyl]thiazole, and biaryl thiophene, thereby treating the subject with the organ-specific T cell mediated autoimmune disease. In some embodiments, the methods described herein include administering abatacept, secukinumab, or infliximab to the subject. The ITK inhibitor can be administered to the subject, e.g., a mammal, e.g., a human, orally, intravenously, or by injection Inhibiting ITK function with BMS509744, ibrutinib, 10n, 2-amino-5-(thioaryl)thiazole, 5 aminomethylbenzimidazole, 2-amino-5-[(thiomethyl)aryl]thiazole, or biaryl thiophene can be particularly helpful as the method can inhibit migration of autoreactive T cells into a target tissue, and migration of alloreactive T cells is unaffected.

Unless otherwise defined, all technical terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, and other references mentioned herein are incorporated by reference in their entirety. In case of conflict, the present specification, including definitions, will control. Other features and advantages of the invention will be apparent from the following detailed description and figures, and from the claims.

### DESCRIPTION OF DRAWINGS

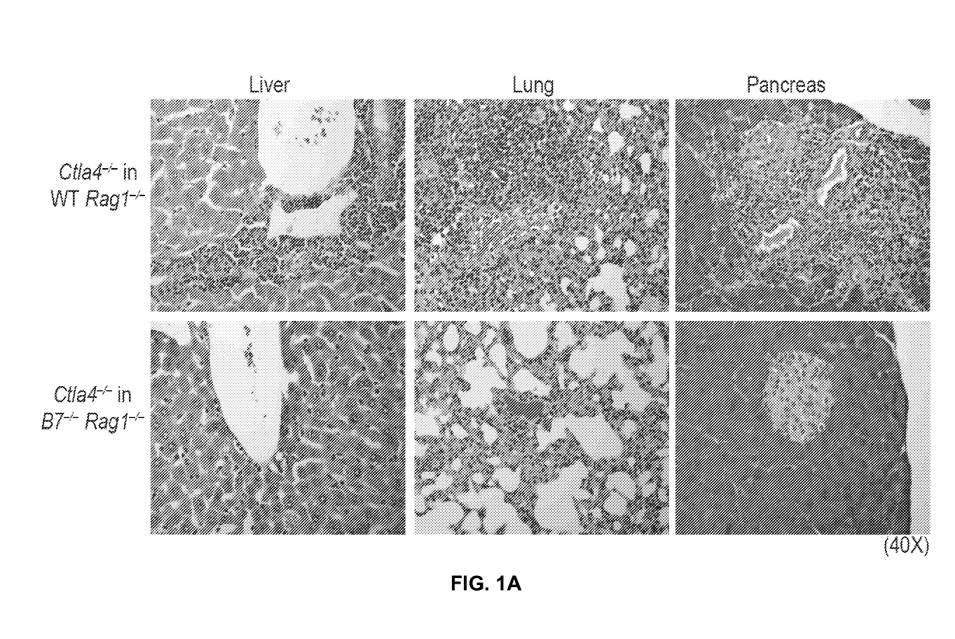

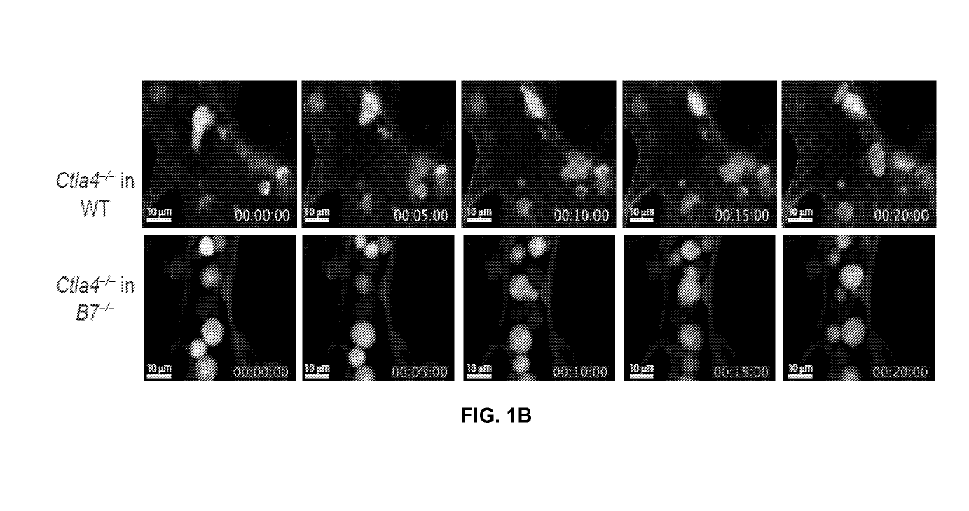

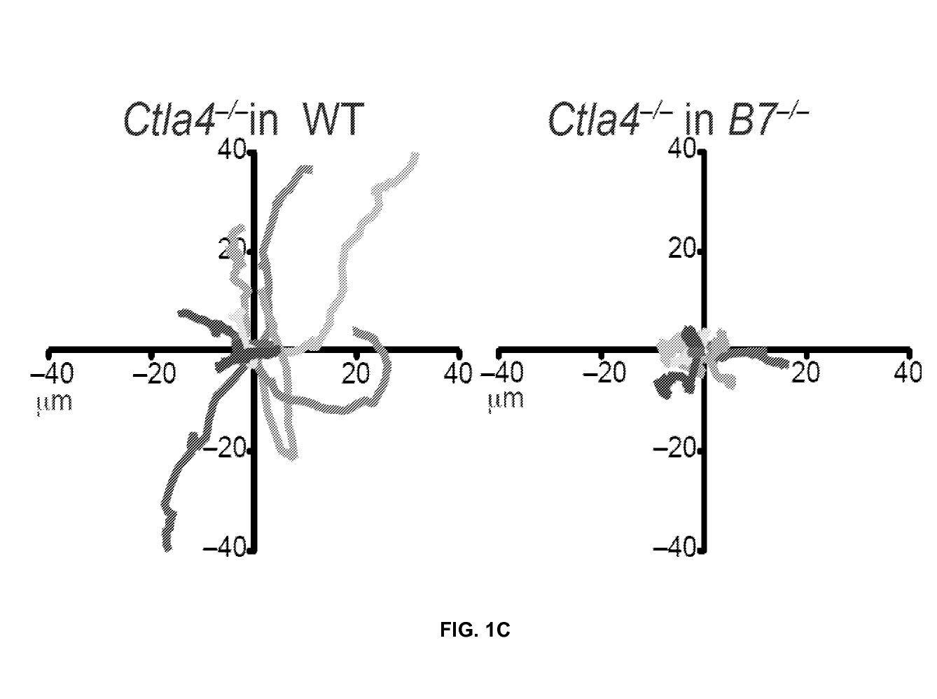

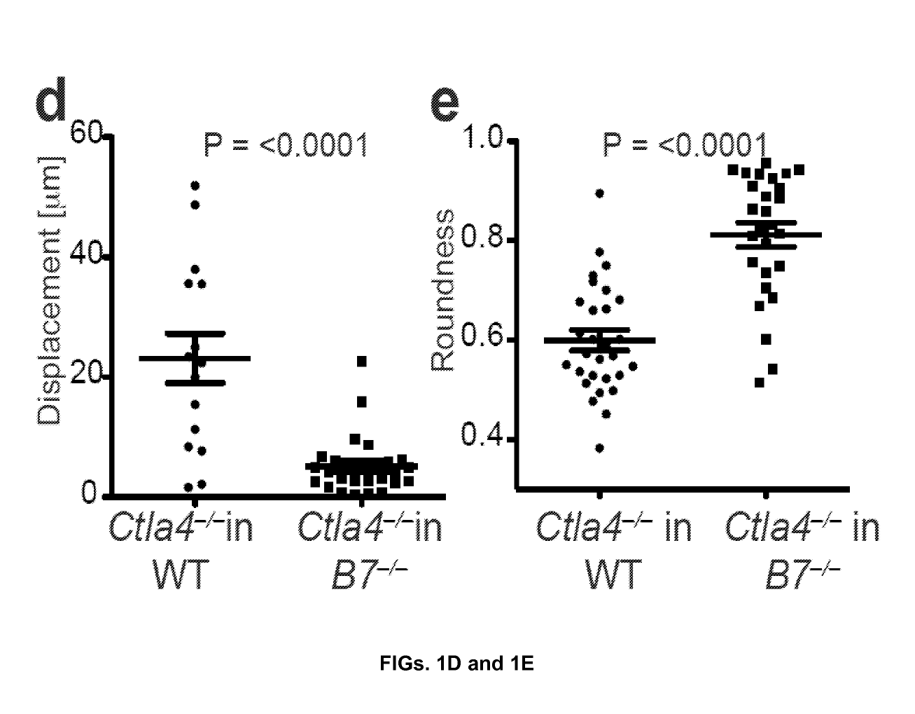

FIG. 1: B7 signals regulate autoreactive Ctla4^{-/-} T cell migration.
A. 20-30×10⁶ LN cells from 3 weeks old Ctla4^{-/-} mice were adoptively transferred into Rag1^{-/-} and B7^{-/-} (Cd80^{-/-}Cd86^{-/-)Rag}1^{-/-} mice. Rag1^{-/-} recipients of Ctla4^{-/-} cells were suffering from a wasting disease starting at 2-3 weeks post transfer and had to be euthanized within 3 wks of the first symptoms. Representative H&E stained sections of indicated organs at 3 wks post-transfer showed massive infiltration by Ctla4^{-/-} T cells into all tissues in B7-sufficient Rag1^{-/-} recipients, but none in B7^{-/-}Rag1^{-/-} recipients. Data are representative of at least 3 experiments with 4-6 mice in each group. B-E. Recipient mice were i.v. injected with fluorescent dye, CMTMR-Orange, to visualize lung vasculature. 30 minutes later, CFSE labeled Ctla4^{-/-} T cells were injected i.v. into either WT or B7^{-/-} mice. B. Representative frames (0-20 minutes) from a Video Savant movie recording showing movement of T cells (green) in blood vessels (red) of lung slices. C. 2-D tracks of 10 representative T cells within blood vessels, superimposed after normalizing their starting coordinates to the origin during 10 minutes of recording. A minimum of at least 30 cells was analyzed for each genotype. Scale: 0.18 uM/pixel. D. Displacement of individual Ctla4^{-/-} T cells in WT or B7^{-/-} lungs from the point of origin in 10 minutes. E. Roundness of cells as calculated by ImageJ (NIH) software. 1=circular object; <1: decreasing circularity. Data in D and E show mean and standard error of means (SEM), p values in D and E are based on the Mann-Whitney and Student's t-test respectively.

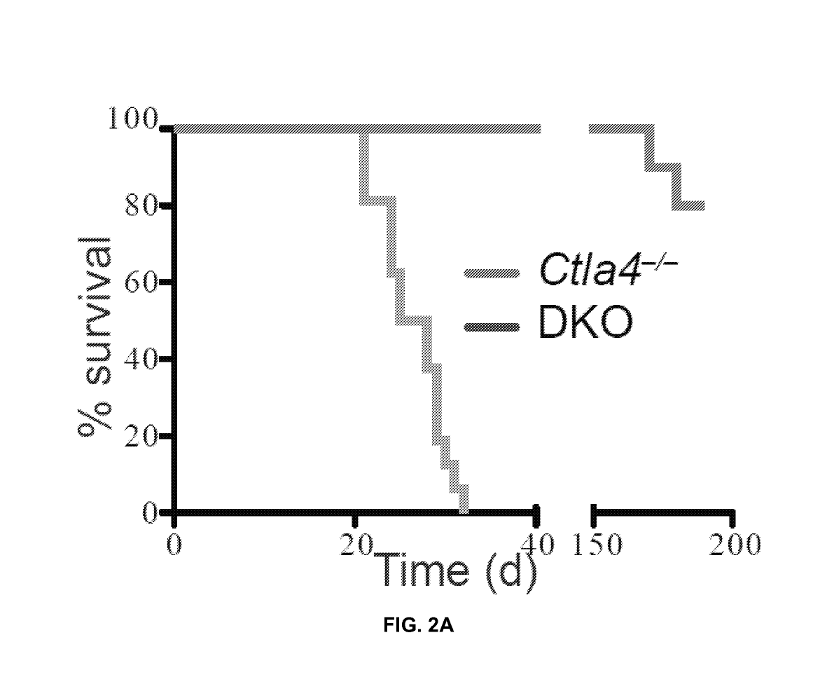

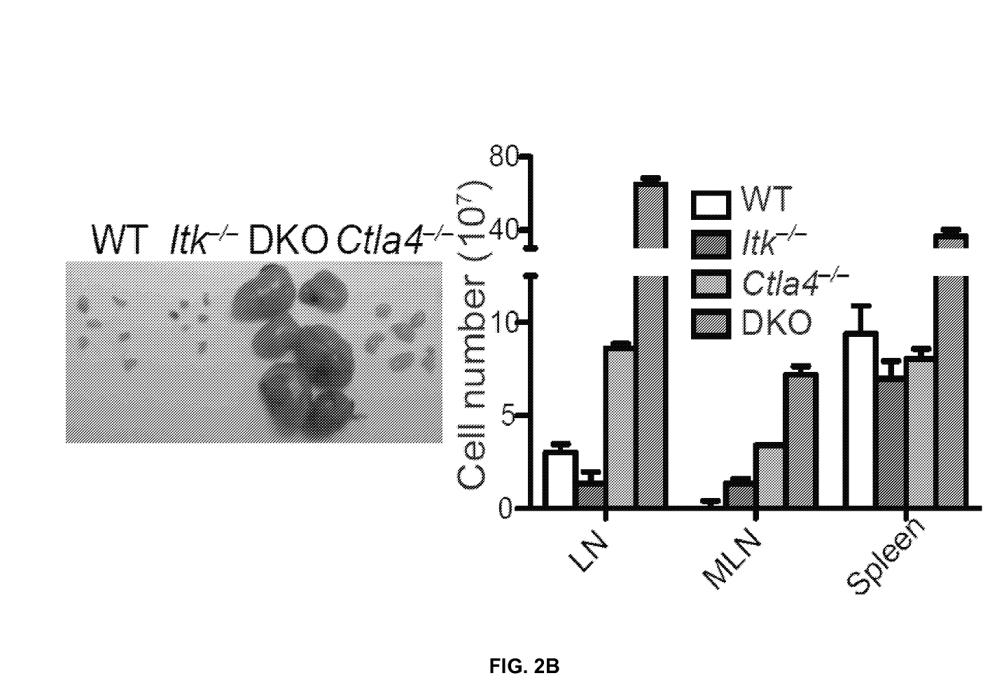

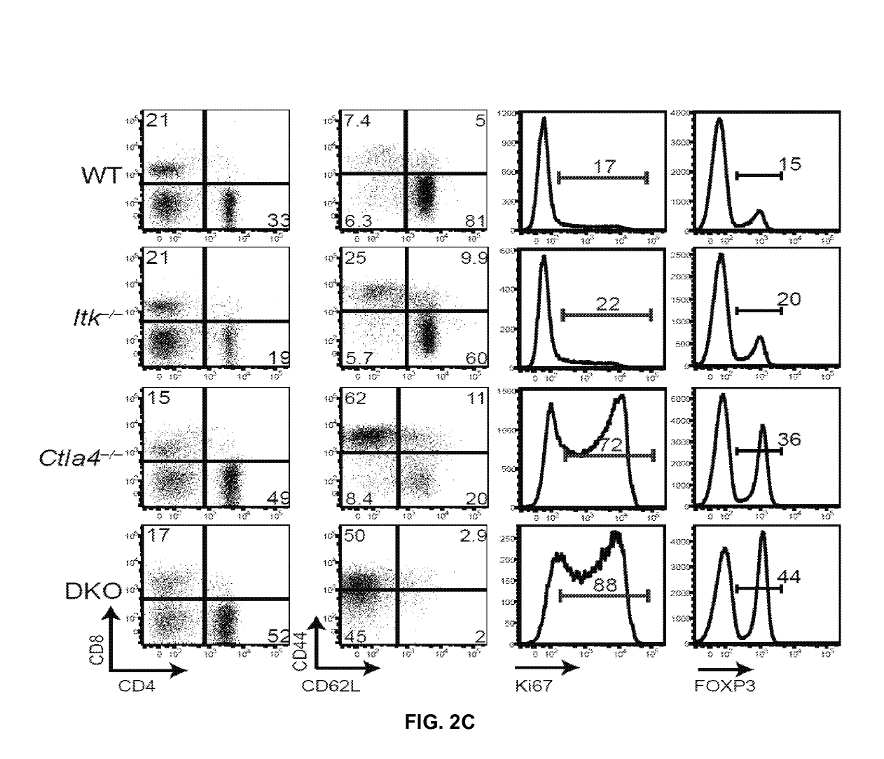

FIG. 2: ITK deficiency prolongs lifespan of Ctla4^{-/-} mice without inhibiting lymphoproliferation.
A. Survival curves of Ctla4^{-/-} (n=10) and Itk^{-/-}Ctla4^{-/-} (DKO) (n=10) mice. P-value =<0.0001 (Log-rank Mantel-Cox test). B. (Left) Size of peripheral (inguinal, axial and brachial) lymph nodes (LN), (Right) Lymphocyte cell numbers in peripheral LNs, mesenteric LNs (MLN) and spleen of 3 weeks old Ctla4^{-/-} and 6-8 weeks old WT, Itk^{-/-} and DKO mice. C. (From left to right) Frequency of CD4⁺ and CD8⁺ T cells, frequency of CD4⁺ conventional T (Tconv) cells expressing activation markers CD44 and/or CD62L, frequency of CD4⁺ Tconv cells expressing Ki67 and frequency of FOXP3⁺ Treg cells in LNs of 3 weeks old Ctla4^{-/-} and 6-8 weeks old WT, Itk^{-/-} and DKO mice. Data in B and C are representative of 3 independent experiments with at least 4 mice in each group.

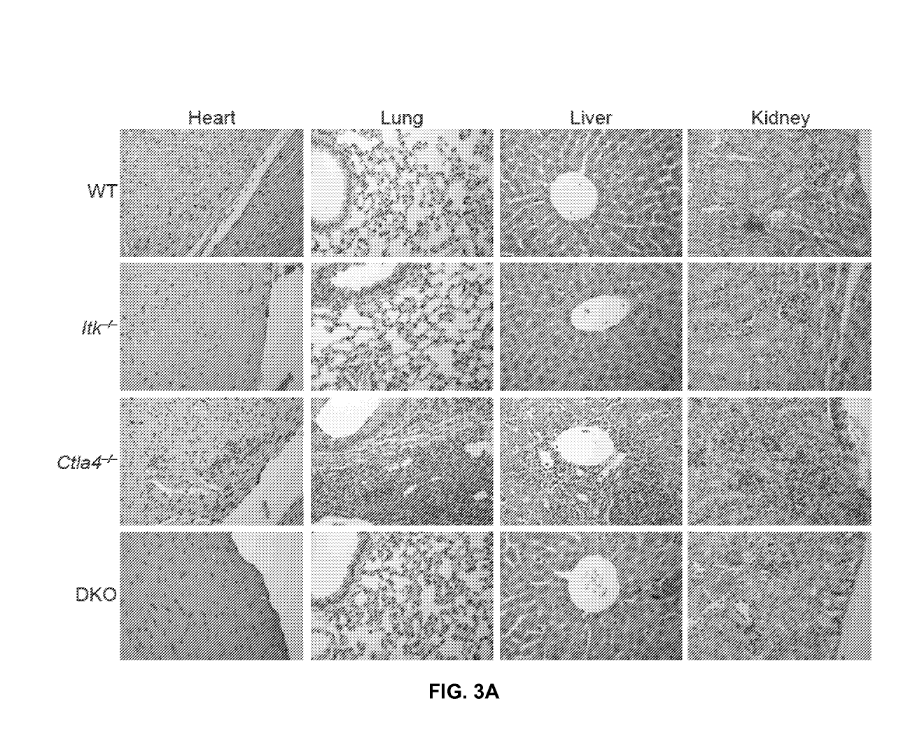

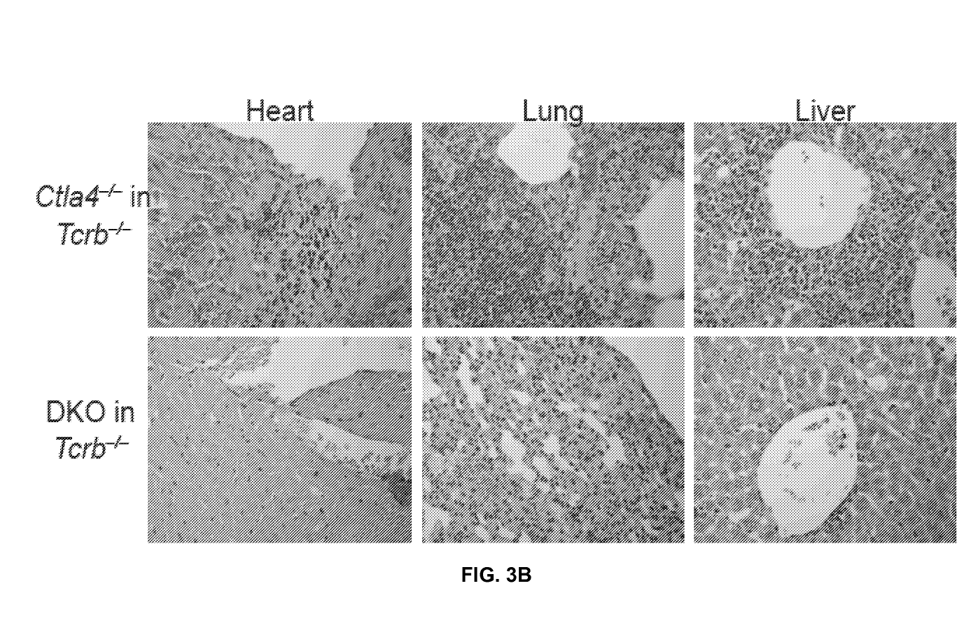

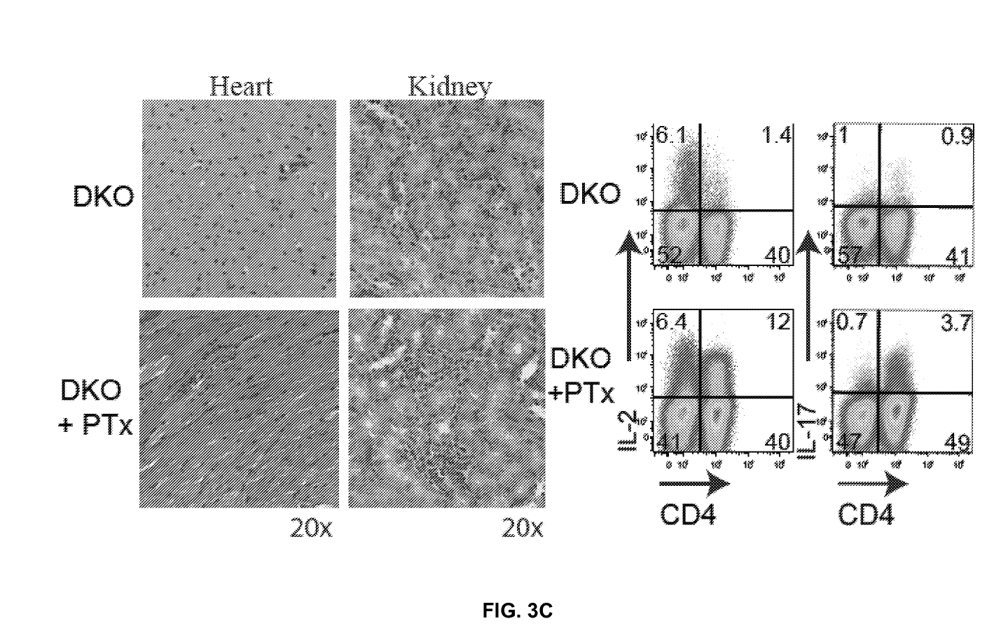

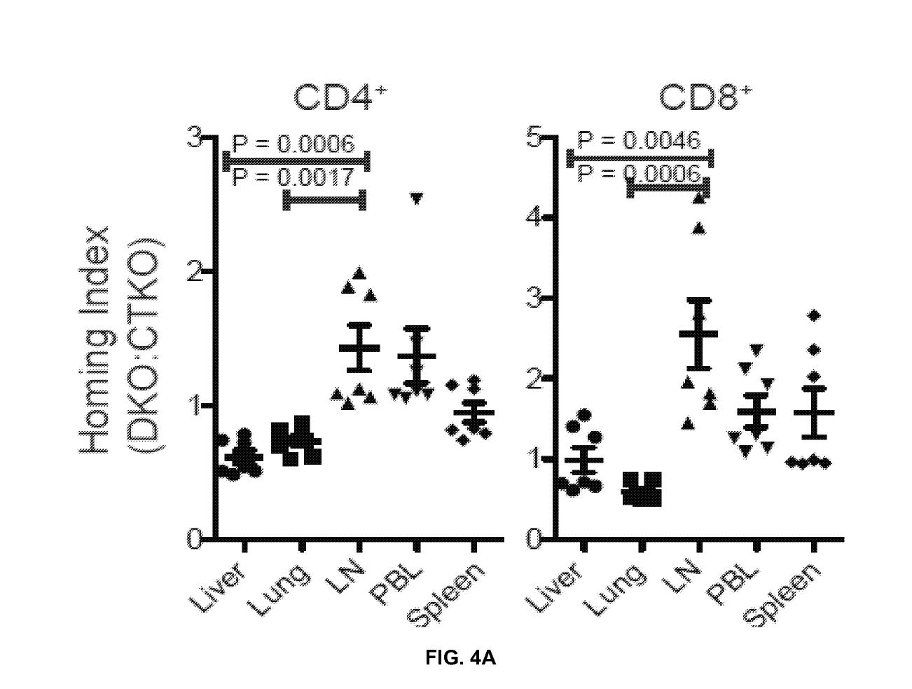

FIG. 3: ITK deficiency prevents Ctla4^{-/-} T cell infiltration into tissues.
A. Representative H&E stained histological sections of indicated organs from 3 weeks old Ctla4^{-/-} and 8-12 weeks old WT, Itk^{-/-} and DKO mice. B. Representative H&E stained histological sections of indicated organs of Tcrb^{-/-} mice 4 weeks after adoptive transfer of 10×10⁶ CD4⁺ T cells from Ctla4^{-/-} and DKO mice. C. 6-8 weeks old DKO mice were given 3 intra-peritoneal injections of pertussis toxin (PTx) on days 0, 2 and 6. (Left) Representative H&E stained histological sections of indicated organs 8 weeks after injections. (Right) Representative flow cytometry profiles of ex vivo IL-2 and IL-17 production by LN CD4⁺ T cells from DKO mice and PTX treated DKO mice. Data in B and C are representative of 3 independent experiments with at least 4 mice in each group.

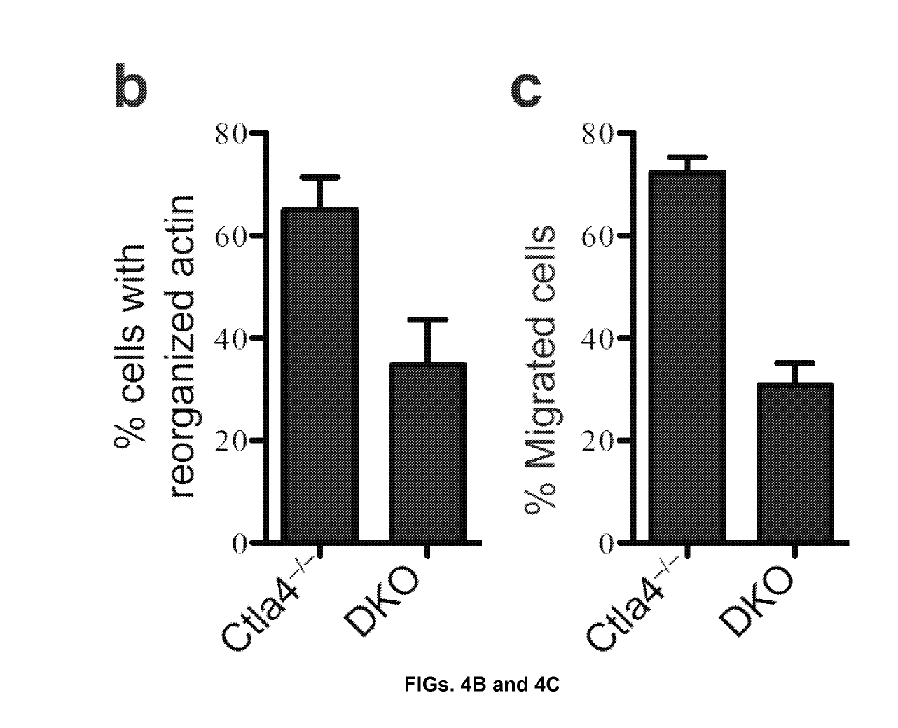

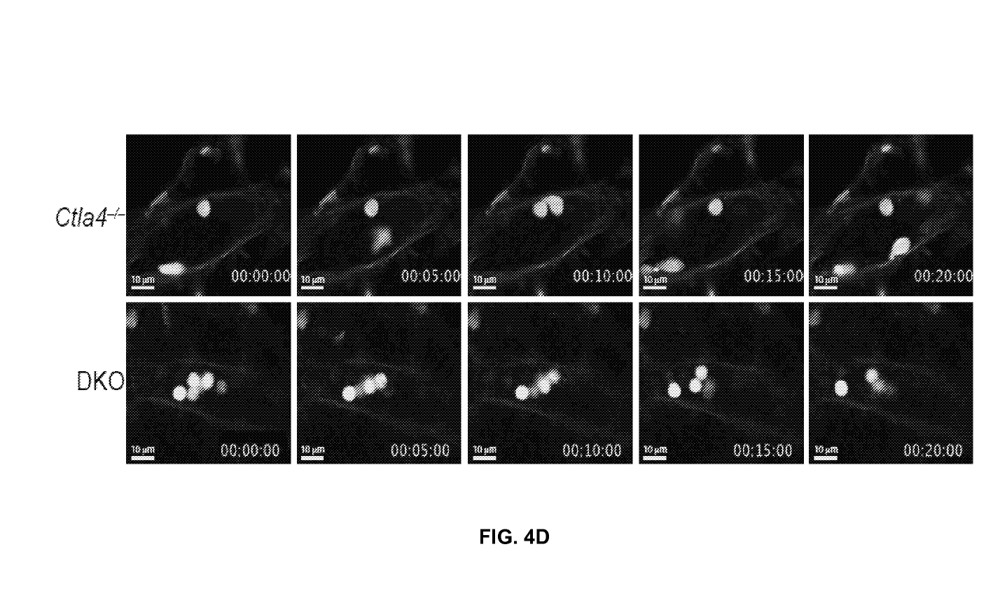

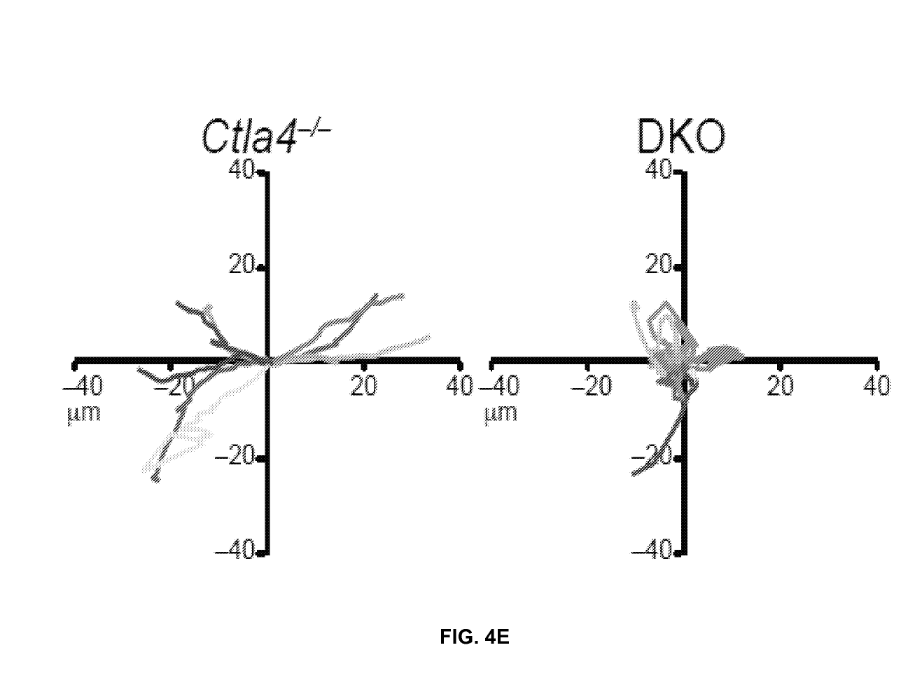

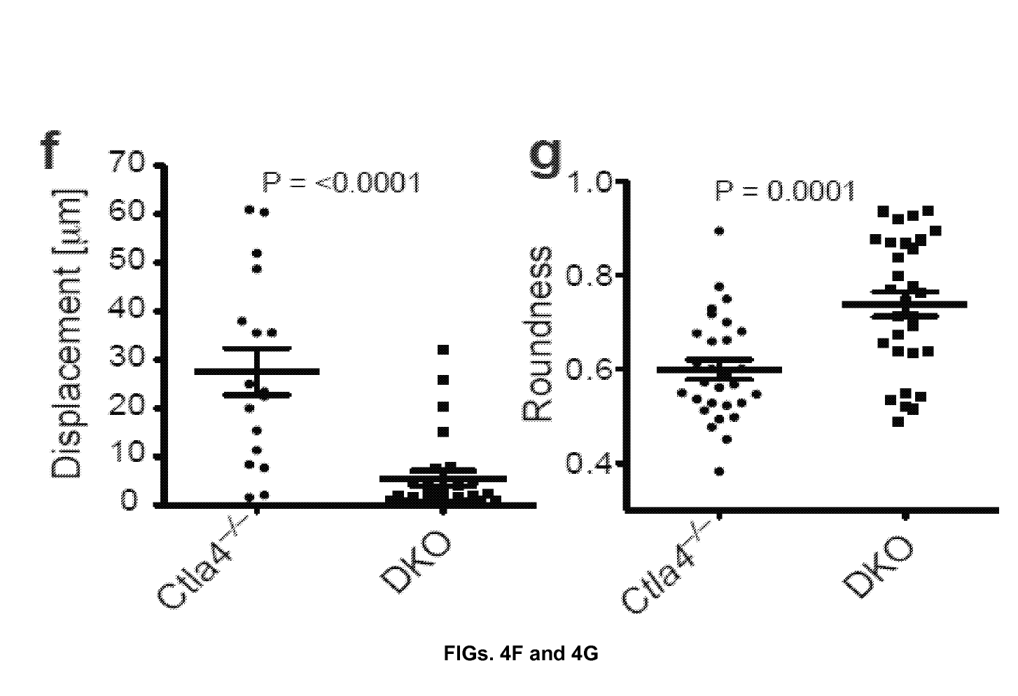

FIG. 4: DKO T cells have defects in trans-endothelial cell migration.
A. Homing index (HI) of eFluor670-labeled Ctla4^{-/-} and CFSE-labeled DKO CD4⁺ and CD8⁺ T cells in indicated organs 6-8 hours after transfer into female Rag1^{-/-} mice. HI was calculated as the ratio of CFSE:eFluor670 in tissues normalized to the ratio of CFSE:eFluor670 CD4⁺ and CD8⁺ T cells injected into the host. Data are combined from two independent experiments, each with 3-4 recipients/group. Similar results were obtained with male recipients, but the presence of female donor cells recognizing male antigens was a confounding factor for CD8⁺ T cells. B. Frequency of CD4⁺ T cells with polarized F-actin in the LNs and spleens of 3 weeks old Ctla4^{-/-} and 8 weeks old DKO mice. Data are from 3 independent experiments with at least 300 cells analyzed for each genotype. C. Frequency of migration of CD4⁺ T cells from Ctla4^{-/-} and DKO mice across an endothelial cell (SVEC4-10 cells; model system to study chemokine, adhesion and costimulatory molecule-regulated diapedesis) layer in vitro. Data are representative of at least 4 independent experiments. D-G. 20-50×10⁶ T cells from LNs of 3 weeks old Ctla4^{-/-} and DKO mice were labeled with CFSE and transferred intravenously into Rag1^{-/-} mice that had been previously injected with CMTMR dye. Movement and behavior of T cells within vasculature of lung slices were observed using 2-photon microscopy. D. Representative frames (0-20 minutes) from a Video Savant movie recording showing movement of T cells (green) in blood vessels (red) of lung slices. E. 2-D tracks of 10 representative T cells within blood vessels, superimposed after normalizing their starting coordinates to the origin during 10 minutes of recording. A minimum of at least 40 cells was analyzed for each genotype. F. Displacement of Ctla4^{-/-} and DKO T cells from the point of origin. G. Shape of Ctla4^{-/-} and DKO T cells within blood vessels in lung slices (1=circular cell, <1=elongated cell). Data in D-G are from 6 independent experiments. Data in A-C and F, G show mean, SEM and p-values based on Student's t-test.

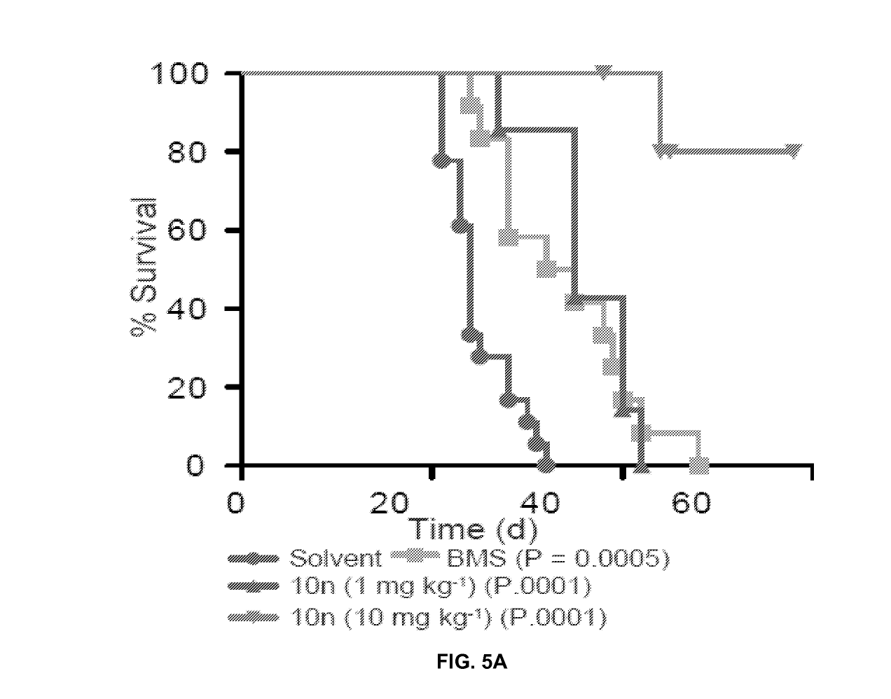

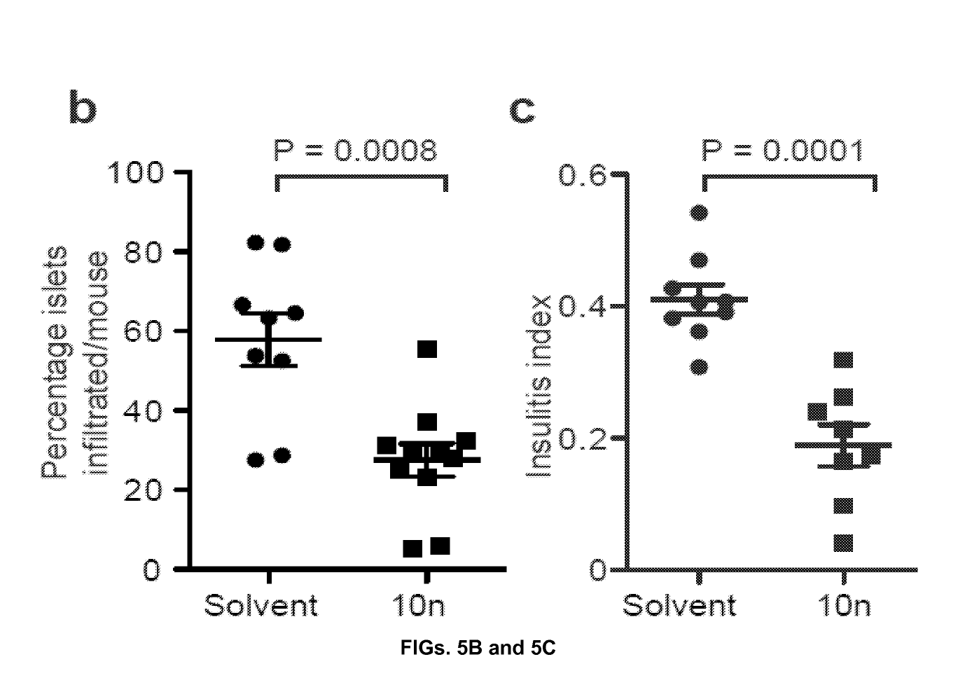

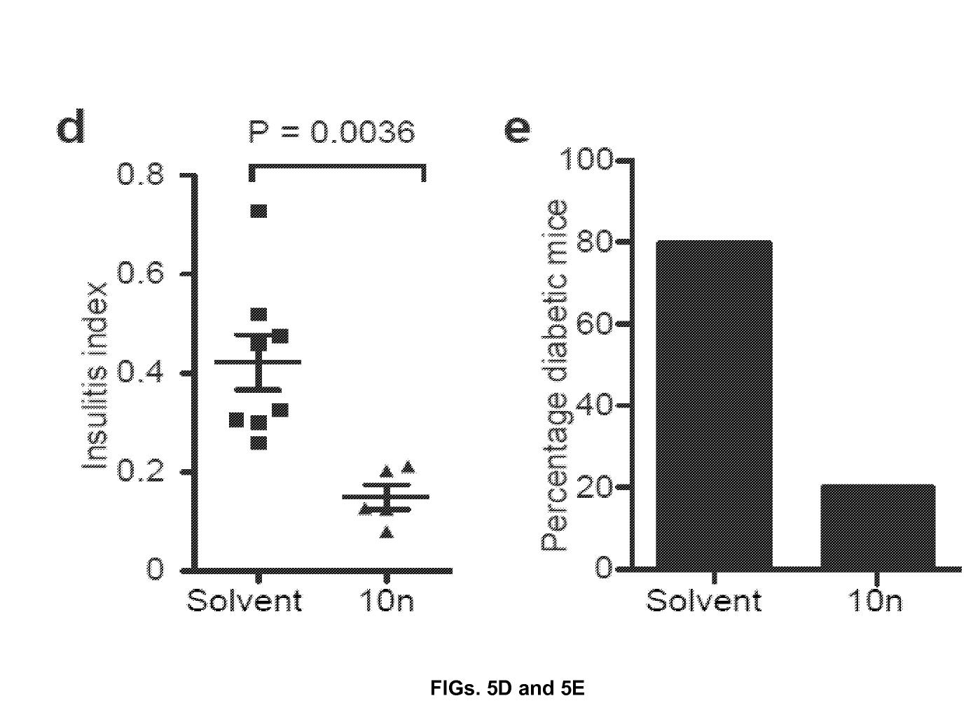

FIG. 5: ITK inhibitors modulate autoimmunity in vivo.
A. Survival curves of Ctla4^{-/-} mice that were treated with ITK inhibitors, BMS509744 at 50 mg/kg (n=11), and 10n at 1 mg/kg (n=8) and 10 mg/kg (n=5), starting at 10 days of age. Control mice (n=10) were injected with solvent alone. P-values were determined using the Log-rank Mantel-Cox test. B. Frequency of islets infiltrated and C. the insulitis index of 8 weeks old female NOD mice that had been treated with ITK inhibitor 10n, at 1 mg/kg from 5 weeks of age. Data are representative of three experiments with 4-10 mice in each group per experiment. D, E. CD4⁺ Tconv cells from BDC2.5/NOD mice were transferred into NOD/Scid mice followed by ITK inhibitor treatment at lmg/kg. D. Insulitis index and E. frequency of diabetic mice. Data are representative of 3 experiments with 10 mice in control group and 5 mice in ITK inhibitor treated group.

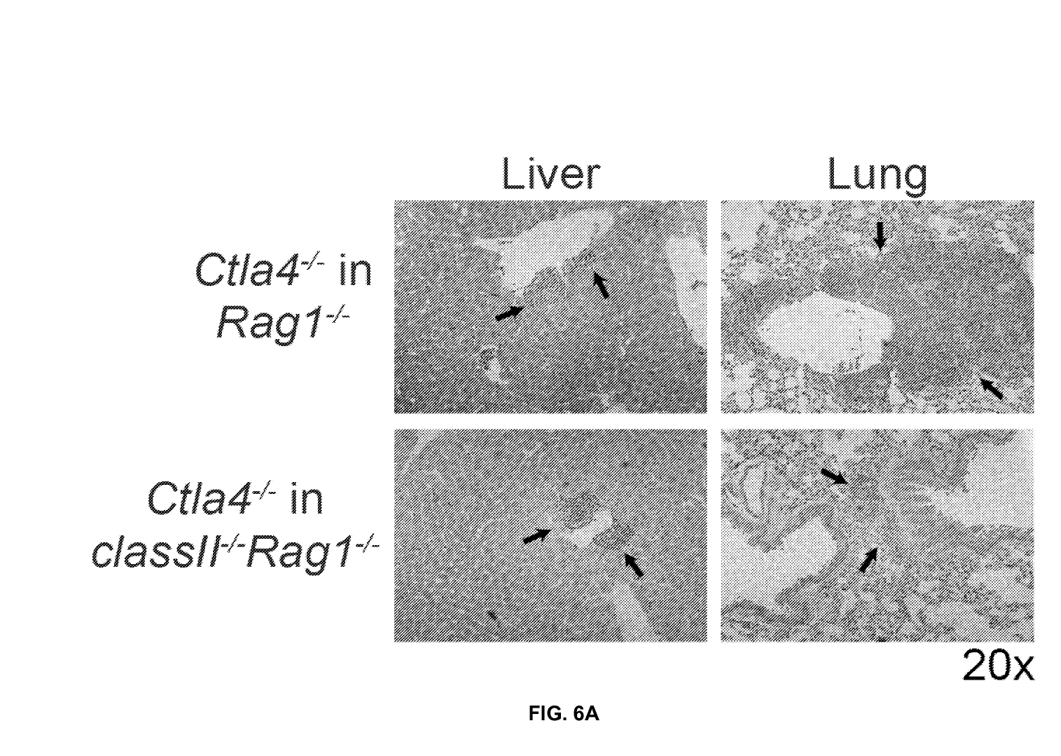

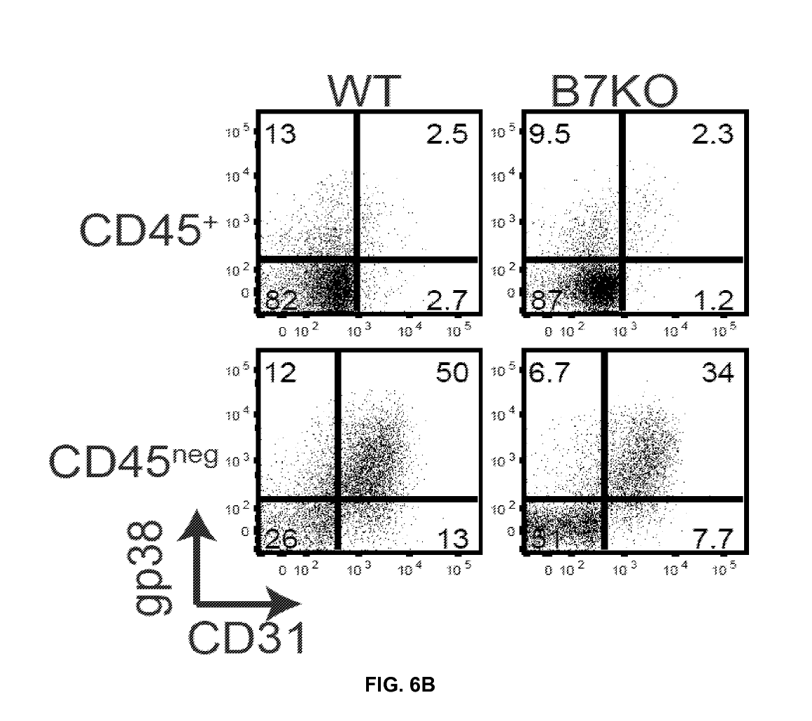

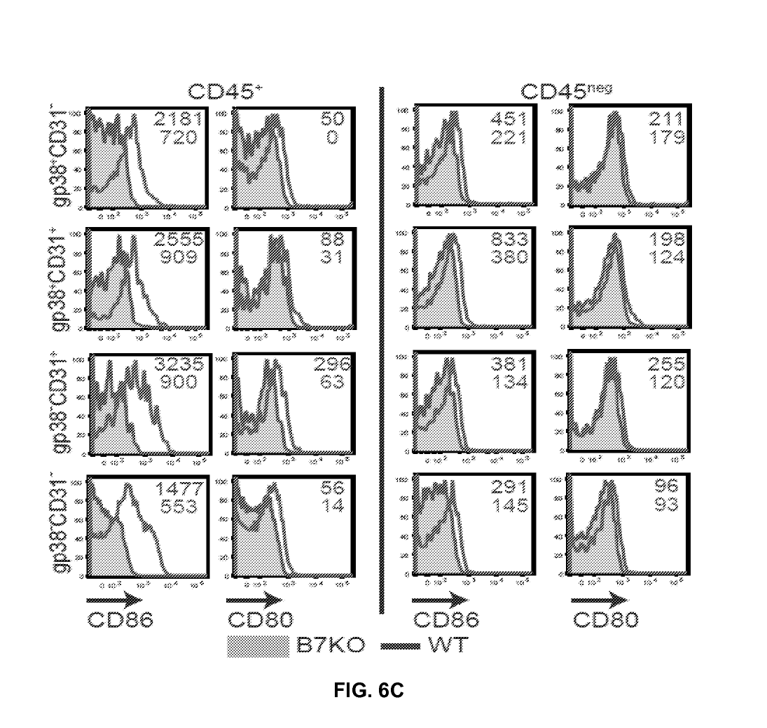

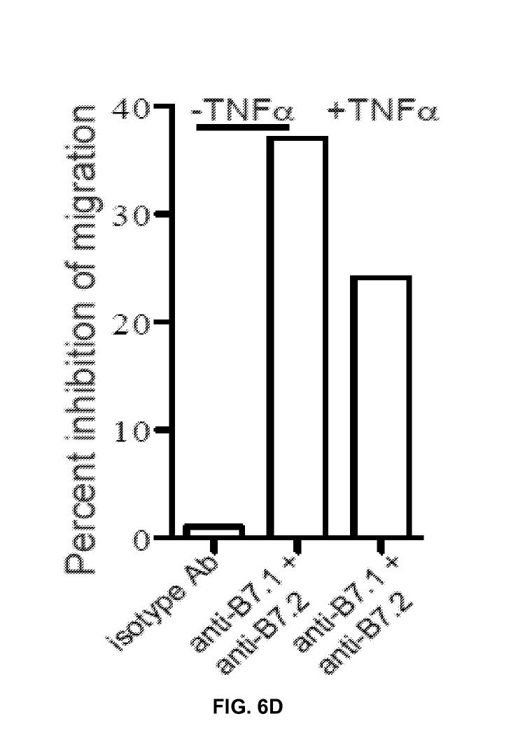

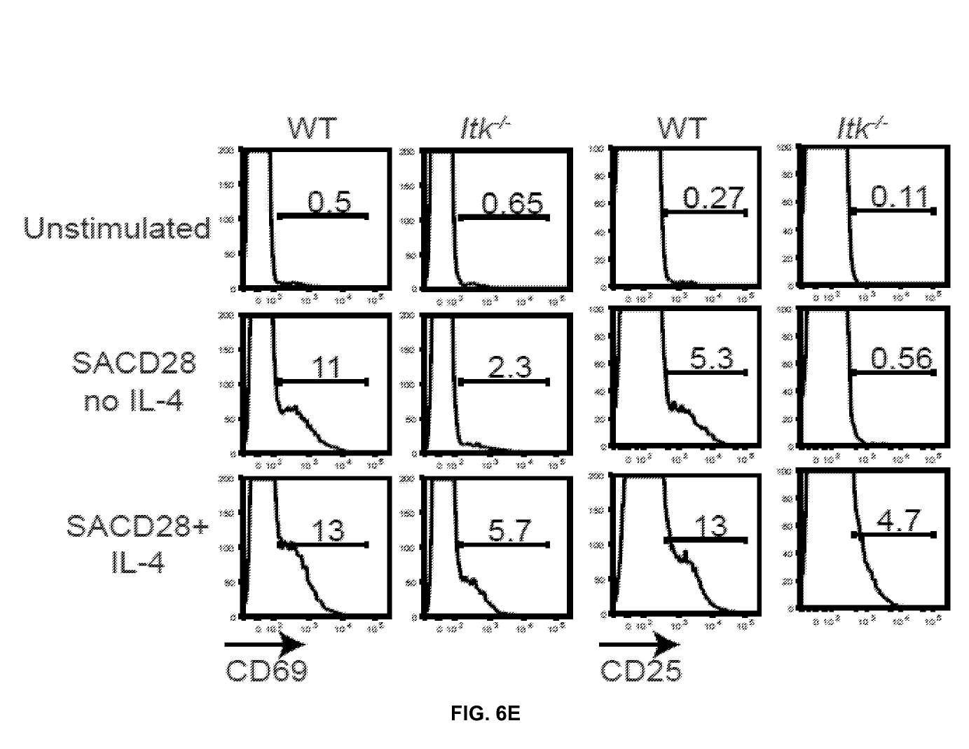

FIG. 6: CD28-B7 interactions regulate trans-endothelial T cell migration.
A. Representative H&E stained sections of indicated organs from Rag1^{-/-} (n=7 for histology; n=10 total analyzed) and MHC ClassII^{-/-}Rag1^{-/-} (n=4; n=12) mice that had received Ctla4^{-/-} cells 6 weeks prior to analysis, respectively. Both Rag1^{-/-} and MHC ClassII^{-/-}Rag1^{-/-} recipients showed infiltration of Ctla4^{-/-} T cells into non-lymphoid tissue, as indicated by arrows. B. Distribution of stromal cell subsets expressing CD45, gp38 (podoplanin) and CD31 in the lungs of 6-8 weeks old C57/BL6 and B7^{-/'1} mice was determined after digestion (9 ml of 1 mg/ml collagenase Type II+1 ml of 2.5 U/ml Dispase in 1×PBS+100 μ. of 1mg/ml solution of DNase I per gram of tissue) at 37° C. for 30 minutes. In the CD45^{neg} compartment, gp38+CD31^{neg} have been identified as follicular reticular cells (FRCs), gp38⁺CD31⁺ cells as lymphatic endothelial cells (ECs) and gp38^{neg}CD31⁺ cells as blood endothelial cells. The CD45^{neg}gp38^{neg}CD31^{neg} cells are a heterogeneous population of cells including vascular pericytes and other stromal cells. CD45⁺ cells expressing gp38 are follicular dendritic cells and macrophages but may also include CD45^{dim} mendotheliai cell subsets. C. Expression of CD80 and CD86 on different stromal cell compartments shown in b. B7-deficient cells were used as negative staining controls (red filed histograms). Numbers in the plots indicate median fluorescent intensities. Data are representative of 3 independent experiments with two mice in each group. D. Percent inhibition of migration of Ctla4^{-/-}T cells across TNFa stimulated and un-stimulated ECs by anti-B7.1 and anti-B7.2 antibodies compared to isotype antibody. EC monolayer on trans-well insert was pre-treated with B7 antibodies for 2 hours prior to the migration assay. Data are representative of 4 experiments with at least 4 replicates in each experiment. E. Sorted naive (CD44⁻CD62L1⁺CD4⁺ cells from WT and Itk-^{-/-} mice were stimulated at 37° C. with CD28 superagonist antibody (SACD28) (10 μg/ml)±rIL-4 (2 ng/ml). Expression of early activation markers CD25 and CD69 were determined on live CD4⁺ T cells after 24 hours of activation. Data are representative of three independent experiments. Activation using strong agonists (anti CD3 and CD28 antibody crosslinking) resulted in substantially higher expression of both markers (>50% positive).

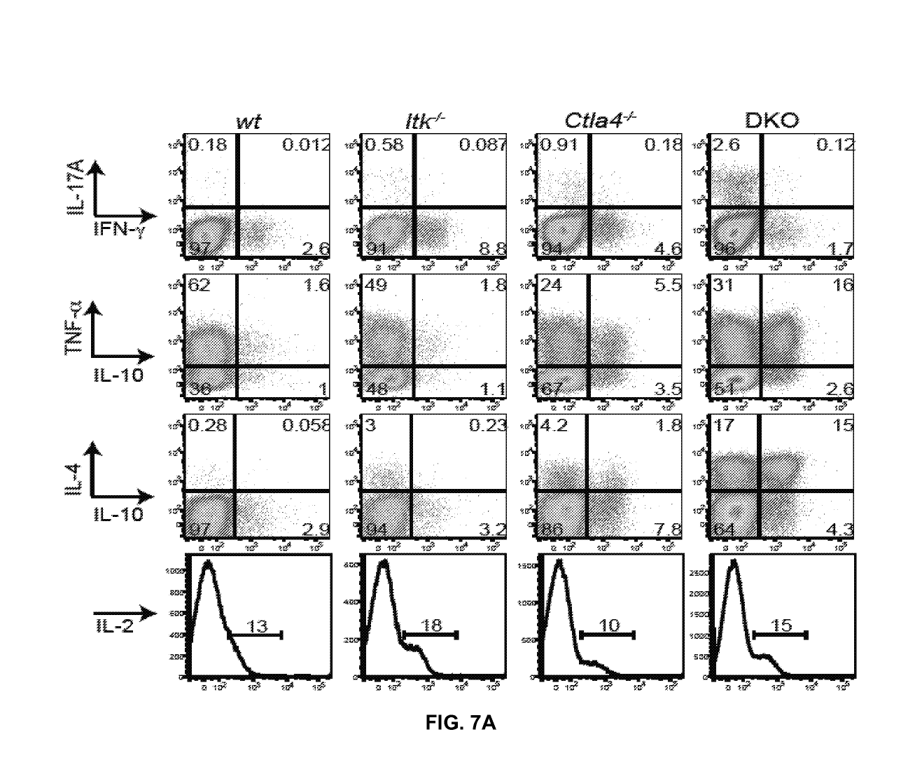

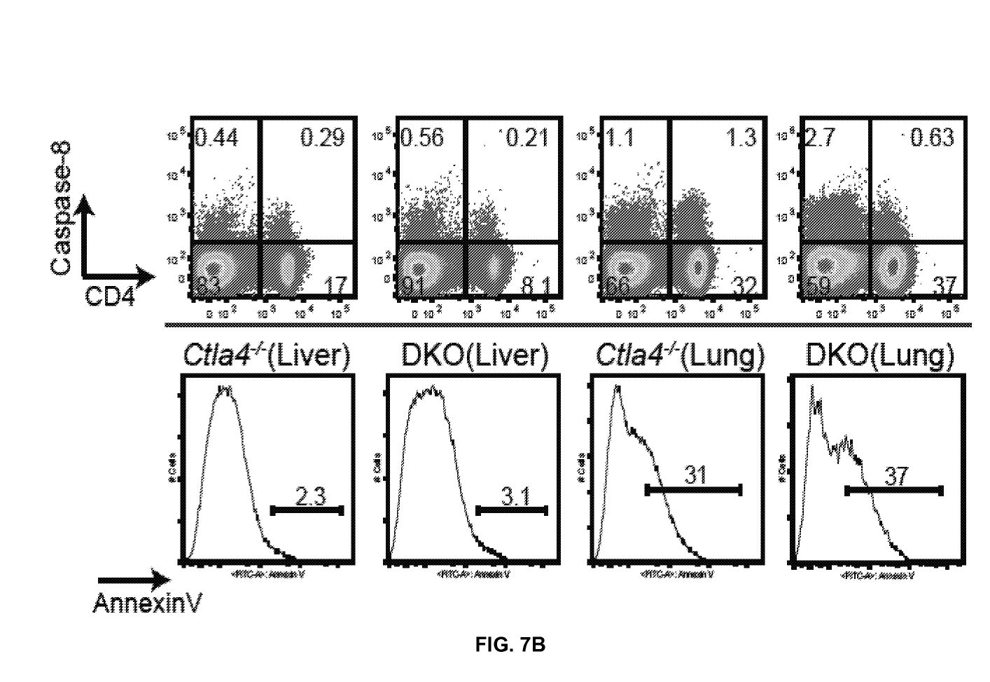

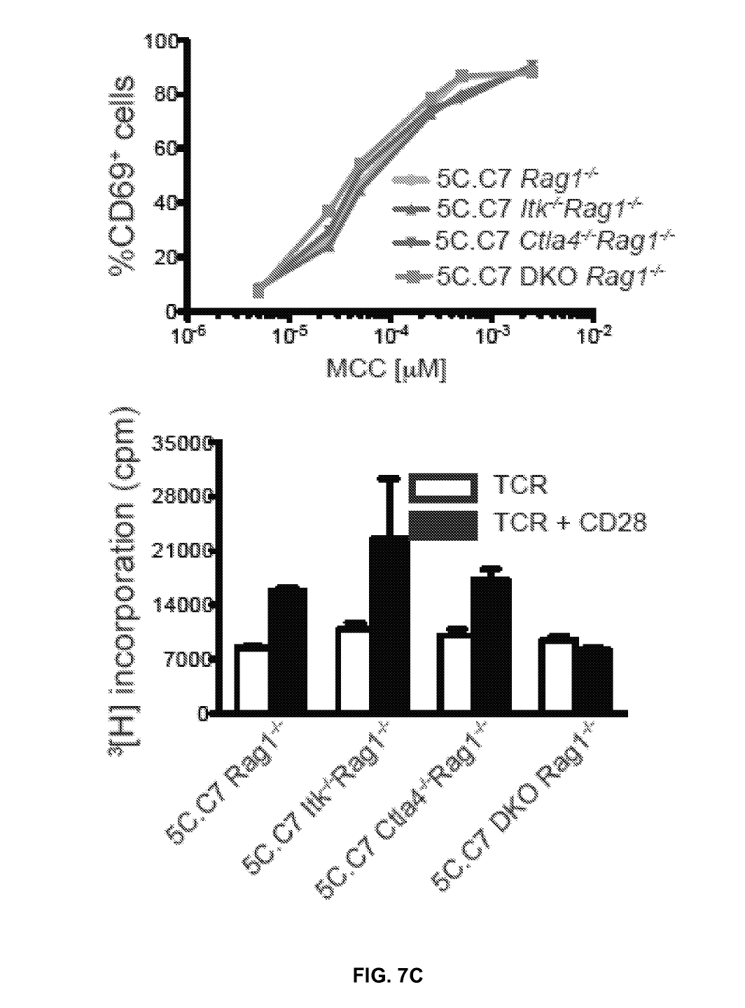

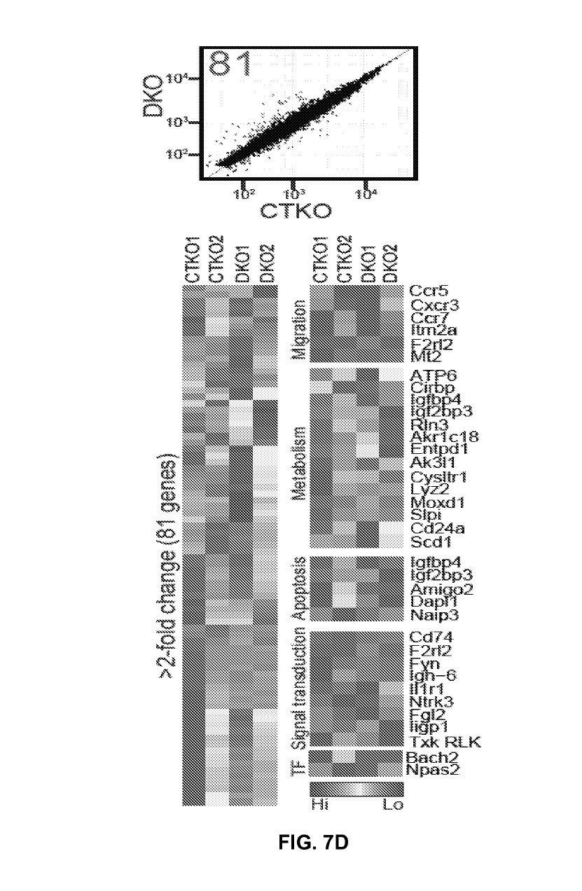

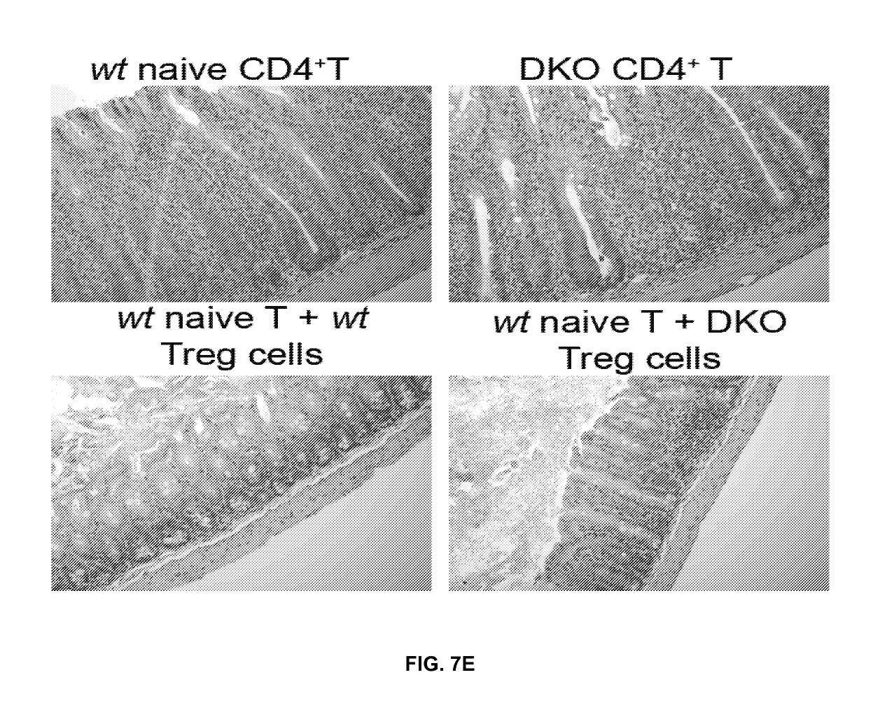

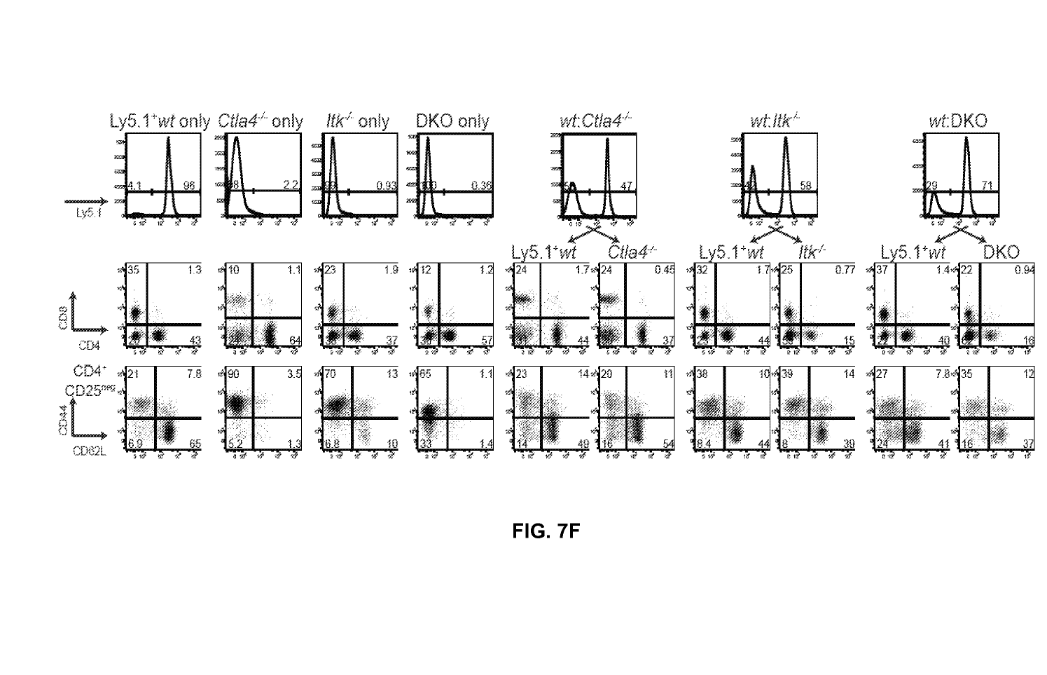

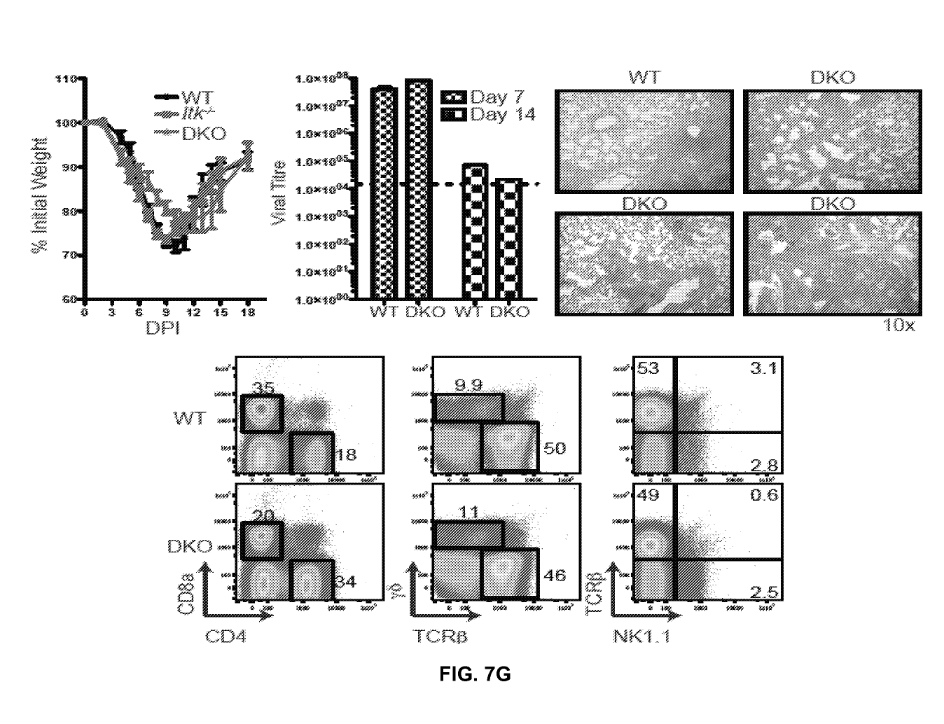

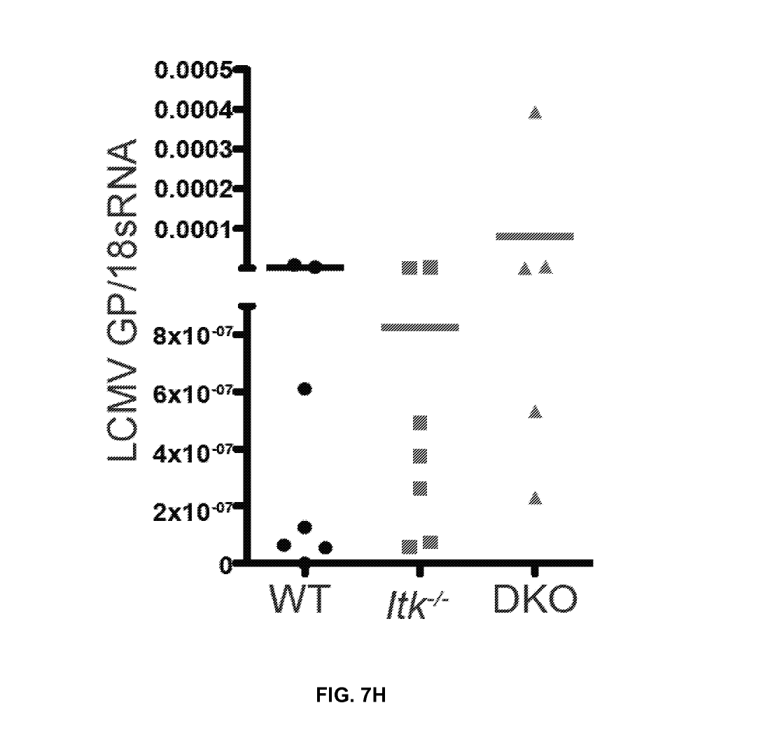

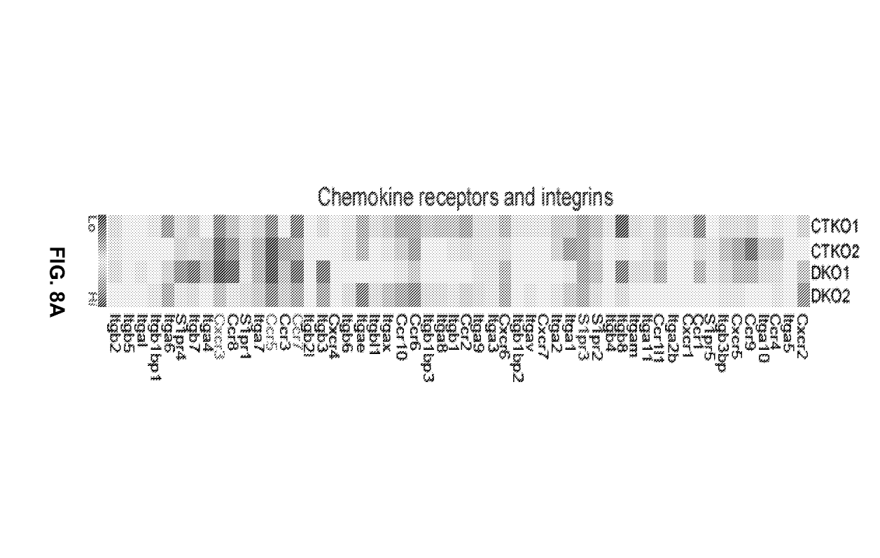

FIG. 7: Phenotypic and molecular characterization of DKO CD4⁺ T cells.
A. CD4⁺ T cells from the LNs of 8 weeks old WT, Itk^{-/-} and DKO mice and 3 weeks old Ctla4^{-/-} mice were tested for their ability to make effector cytokines following 4 hour ex vivo stimulation with PMA and Ionomycin in the presence of Golgi inhibitors. Representative FACS profiles for intra-cellular staining for IL-17A, IFNγ, TNFa, IL-10, IL-4 and IL-2 on CD4⁺ T cells are shown. Data are representative of at least three independent experiments with two mice in each group. While 8 weeks old DKO T cells consistently produced more IL-4 and IL-10 compared to much younger and sick Ctla4^{-/-} mice, the difference was not dramatic when 3 weeks old DKO mice were compared. B. (Top) Expression of the cell death marker Caspase-8 on CD4⁺ T cells from 8 weeks old WT, Itk^{-/-} and DKO mice and 3 weeks old Ctla4^{-/-} mice. (Bottom) Annexin V staining of Thy1.2⁺CD4⁺ T cells in the liver and lungs of 3 weeks old Ctla^{-/-} and 6 months old DKO mice. Older DKO mice were used to obtain sufficient numbers of T cells for analysis. Data are representative of two experiments with 3-4 mice per group. C. 5C.C7Itk^{-/-}Rag1^{-/-} mice were crossed with 5C.7Ctla4^{-/-}Rag1^{-/-} mice to generate 5C.C7DKORag1^{-/-} mice. Naive CD4⁺ T cells were activated with indicated concentrations of moth cytochrome c peptide (MCC) pulsed I-E^{k} expressing CHO cells. (Top) Frequency of T cells expressing the early activation marker CD69 at 24 hours post-activation. (Bottom) Proliferation of T cells activated with 5e^{-4 μ}M was measured by tritiated Thymidine uptake assay. Data are representative of two independent experiments. Error bars are standard error of means. D. Global gene expression profiling of CD4⁺CD25^{neg} T cells from 3 weeks old Ctla4^{-/'1} and DKO mice was performed using an Affymetrix MoGene 1.0 ST array. (Top) Scatter plot showing differential expression of genes between Ctla4^{-/-} (CTKO) and DKO mice. Each dot represents one gene (mean of all probe sets) and red indicates genes with expression change of more than two-fold. Of the 17,552 annotated genes with expression values greater than 120 in at least one subset (classified as expressed genes), 81 genes were changed in expression by greater than 2-fold between Ctla^{-/-} and DKO mice. (Bottom). Heat maps of expression of genes with greater than 2-fold change overall (Left) and segregated based on indicated biological processes (Right). Each column represents individual samples of indicated genotype. All data were log transformed and row normalized for display. 12/81 differentially expressed genes were Ig transcripts that were higher in DKO samples, possibly due to a down regulation of Bach2 in DKO T cells, and are not likely to be functionally relevant for T cells. E. Representative H&E stained sections of colons from indicated mouse groups 6 weeks after adoptive transfer of naïve CD4⁺ T cells with WT or DKO Treg cells. Transmural inflammation and pathology in recipients of naïve WT cells and DKO CD4⁺CD25^{neg} cells alone was observed. Co-transfer of WT Treg cells prevented the development of colitis by WT naive cells, while DKO Treg cells failed to do so. However, the degree of colonic pathology was decreased in the presence of DKO

Treg cells, suggesting that, similar to Ctla4^{-/-} Treg cells, DKO Treg cells might retain some functionality in this assay of Treg cell function. Data are representative of two experiments with four mice in each group. F. (Top) Rag1^{-/-} mice reconstituted with BM from Ly5.1⁺WT, Ly5.2⁺Ctla4^{-/-}, Ly5.2⁺Itk^{-/-} and Ly5.2⁺DKO mice. (Bottom) Rag1^{-/-} mice reconstituted with a 1:1 mix of BM from Ly5.1⁺WT and Ly5.2⁺Ctla4^{-/-} mice, Ly5.1⁺WT and Ly5.2⁺Itk^{-/-} mice or Ly5.1⁺WT and Ly5.2⁺DKO mice. The frequency of CD4+ and CD8⁺ T cells and frequency of activated CD44^{hi}CD62L^{lo}CD4⁺CD25^{neg} T cells in the spleen was determined by flow cytometry at 8 weeks post-reconstitution. Data are representative of two independent experiments with five mice in each group. G. (From left to right) Body weights of the A/PR8 Influenza A virus infected mice at indicated days post-infection (DPI); Lung viral titers were determined at d7 and d14 post-infection (dashed line represents the limit of detection); Representative H&E stained sections of lungs from 1 WT and 3 DKO mice 22 days post-infection; Representative flow cytometry dot plots showing frequency of various T cell subsets in lungs of infected WT and DKO mice. These data indicate that DKO mice clear Flu virus with similar kinetics as WT mice. Further, contrary to the lack of migration of autoreactive DKO T cells in uninfected mice, virus-induced effector T cells in infected DKO mice migrated efficiently to lung tissues. H. qPCR analysis of LCMV glycoprotein expression in spleens from d11 infected mice showed no significant differences in viral clearance between WT and DKO mice. Data in g and h are representative of two independent experiments for each virus infection.

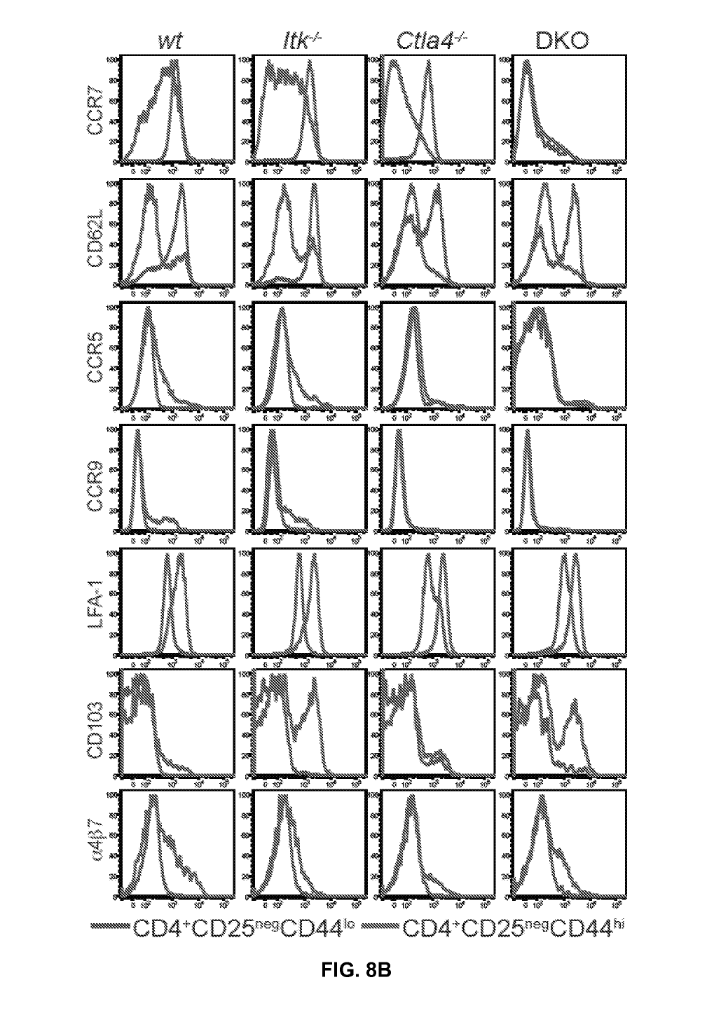

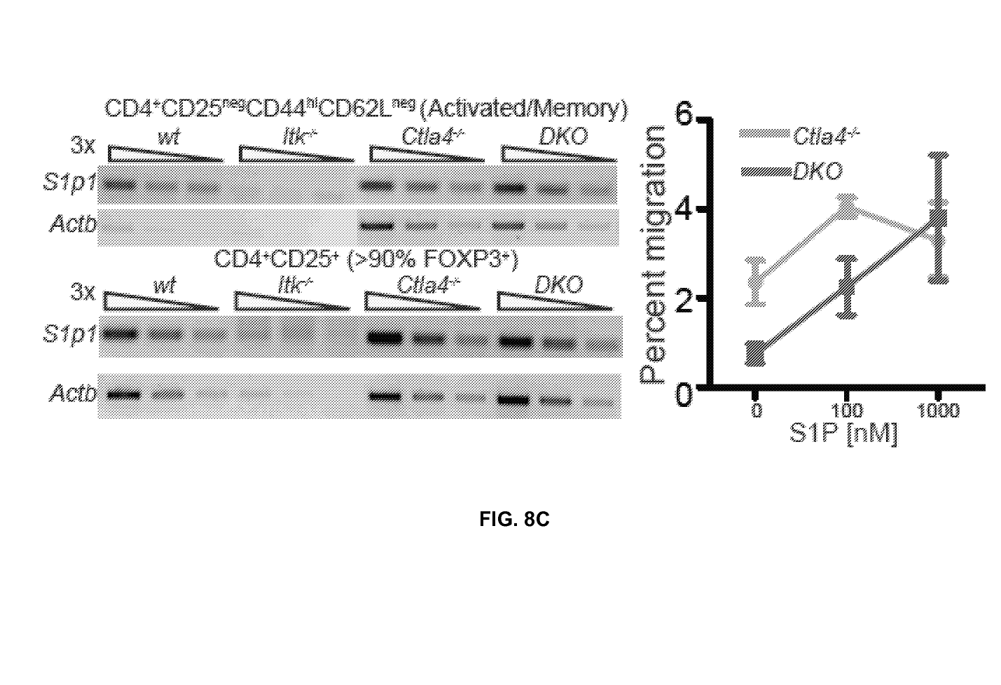

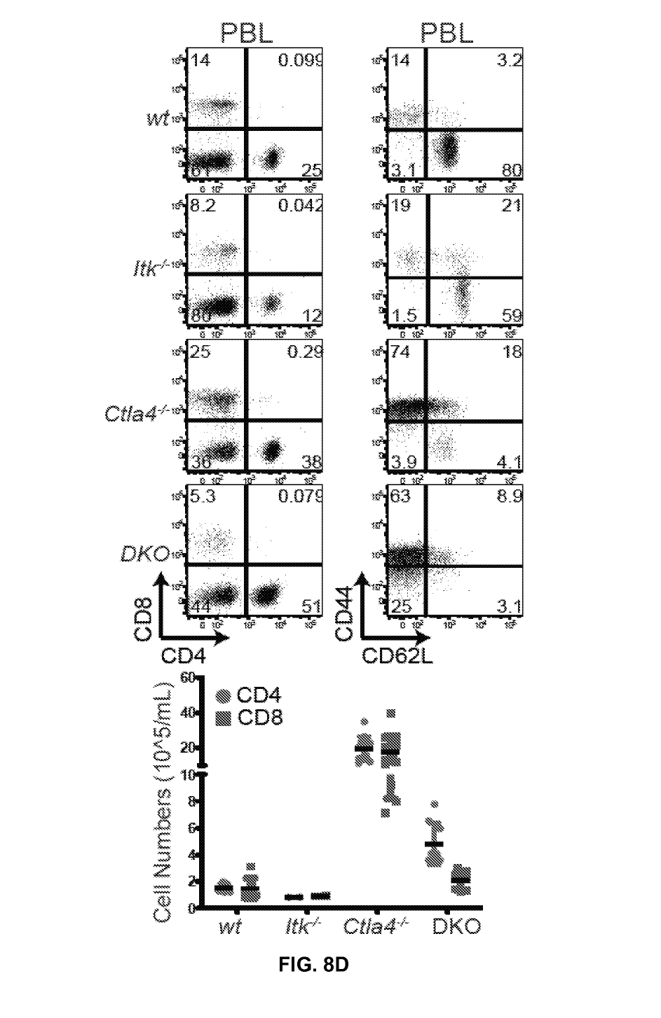

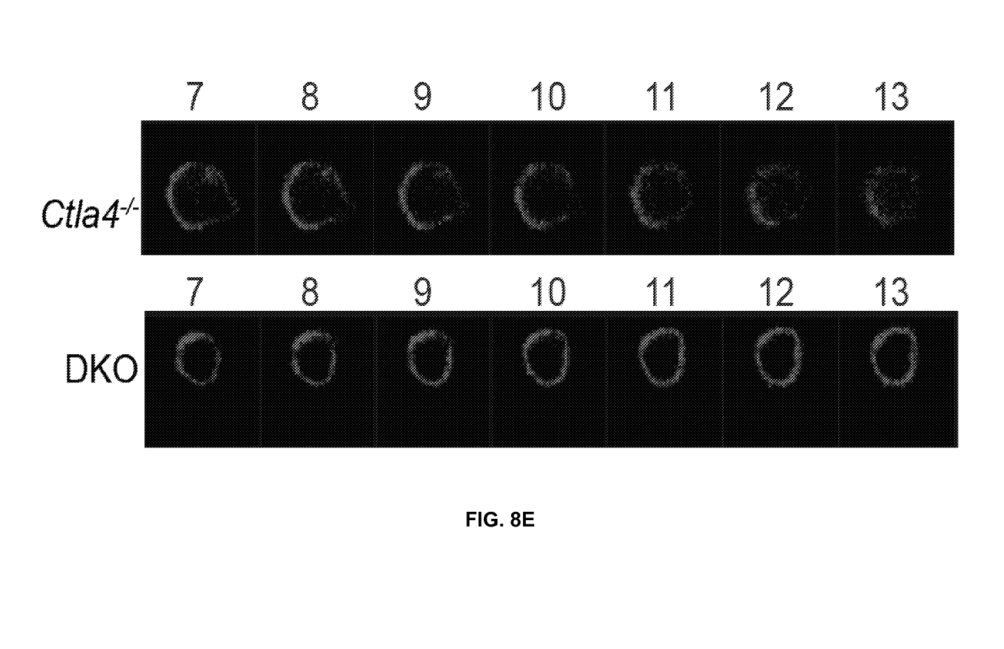

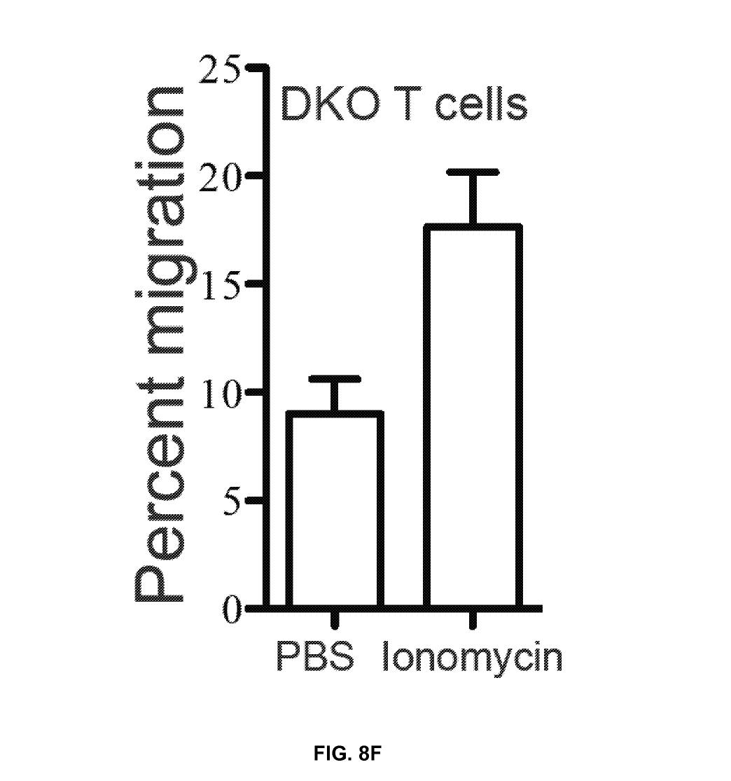

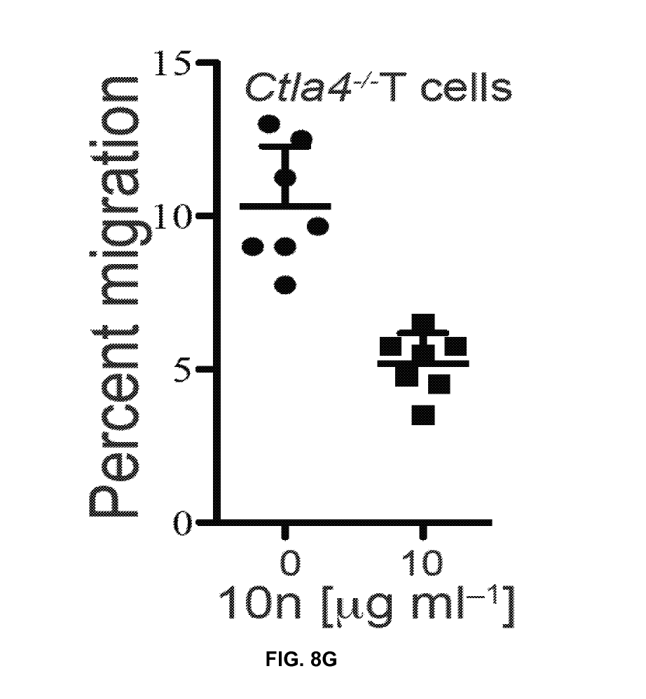

FIG. 8: Similar pattern of chemokine receptor and integrin expression on DKO and Ctla4^{-/-} CD4⁺ T cells.
A. A heat map of gene expression of chemokine receptors and integrins in CD4⁺CD25^{neg} T cells from Ctla4^{-/-} (CTKO) and DKO mice. Data were log transformed and row normalized for display. Each column represents individual samples of indicated genotype. While-transcripts for Ccr5 and Ccr7 were reduced in DKO T cells, protein expression analysis by flow cytometry (b) did not show significant differences to activated (CD44⁺) Ctla4^{-/-} conventional CD4⁺ T cells (Tconv, FOXP3′g) for CCR7 and only a marginal difference in MFI for CCR5. Further, DKO and Ctla4^{-/-} T cells migrated similarly to chemokine CCL5, a ligand for CCR5, suggesting that these pathways were not relevant in the impaired migration of DKO T cells in vivo. B. LN cells from 6-8 weeks old WT, Wand DKO mice and 3 weeks old Ctla4^{-/-} mice were analyzed for the cell surface expression of various chemokine receptors and integrins. Representative flow cytometric histograms showing expression of CCR7, CD62L, CCR5, CCR9, LFA-1, CD103 and α4β7 on CD4⁺CD25^{neg}CD44^{lo} (red) and CD4⁺CD25^{neg}CD44^{hi}(blue) T cells. Low CCR7 expression on CD44^{lo} DKO CD4⁺ T cells relative to those from Ctla4^{-/-} mice reflects a near absence of CD44⁻CD62L⁺ naive T cells in 6 weeks old DKO mice (FIG. 2C). Data are representative of 2 independent experiments with 2-3 mice in each group. C. (Left) Semi-quantitative RT-PCR analysis of expression of S1P1 relative to β-actin in CD4⁺CD25^{neg}CD44^{hi}CD62L^{lo} (activated Tconv) and CD4⁺CD25⁺ (Treg) cells from WT, Itk^{-/-}, Ctla4^{-/-} and DKO mice. (Right) Trans-well migration assay showing frequency of migration of CD4⁺T cells from Ctla4^{-/-} and DKO mice to different concentrations of SIP ligand. Data are representative of 3 independent experiments. D. (Left) Frequency of CD4⁺ and CD8⁺ T cells in peripheral blood lymphocytes (PBL) and (Right) expression of the activation markers CD44 and CD62L (L-Selectin) on blood CD4⁺CD25^{neg} T cells of 6-8 weeks old WT, Itk^{-/-} and DKO mice and 3 weeks old Ctla4^{-/-} mice. (Bottom) Numbers of CD4⁺ and CD8⁺ lymphocytes in peripheral blood of 3 weeks old WT, Itk^{-/-} Ctla4^{-/-} and DKO mice. E. β-actin staining of purified CD4⁺ T cells from 3 weeks old Ctla4^{-/-} and 8 weeks-old mice. Representative serial z-stacks of a single lymphocyte from Ctla4^{-/-} and DKO mice are shown. >85% of CD4⁺T cells had an activated CD44^{hi}CD62L^{lo} phenotype from both Ctla4^{-/-} and DKO mice prior to staining. F. DKO LN cells were stimulated with the Ca²⁺ ionophore, Ionomycin (2 μg/ml) for 2 hours prior to a trans-endothelial migration assay. Plot shows frequency of DKO cells stimulated with Ionomycin or left untreated (in saline) migrating across SVEC4-10 cells after 4 hours. Data are representative of 3 independent experiments with 4-5 replicates in each experiment. G. CD4⁺T cells from Ctla4^{-/-} mice were pre-treated for 2 hours with the ITK inhibitor 10n (10 μg/ml) prior to a trans-endothelial migration assay Error bars are standard error of means. Data are representative of at least two independent experiments with 3-4 replicates for each condition.

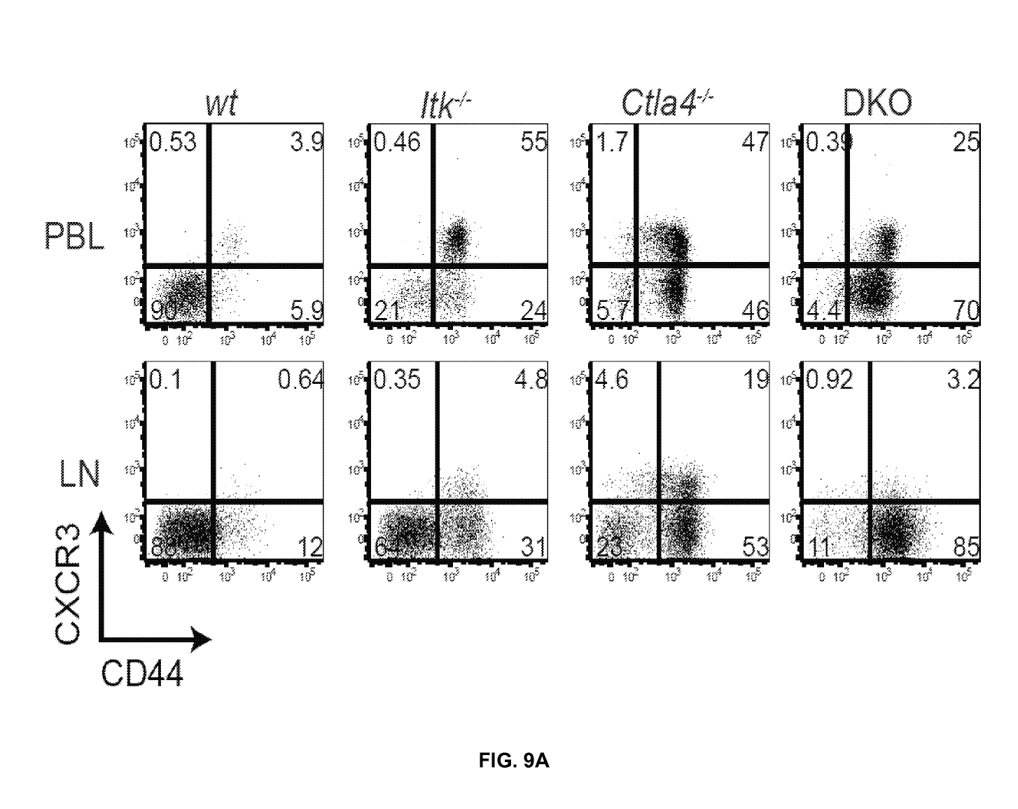

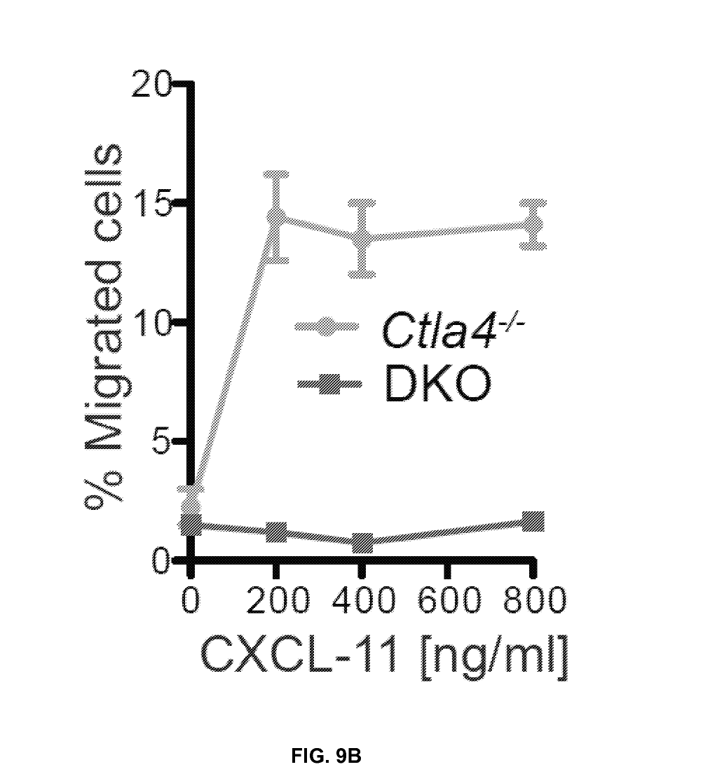

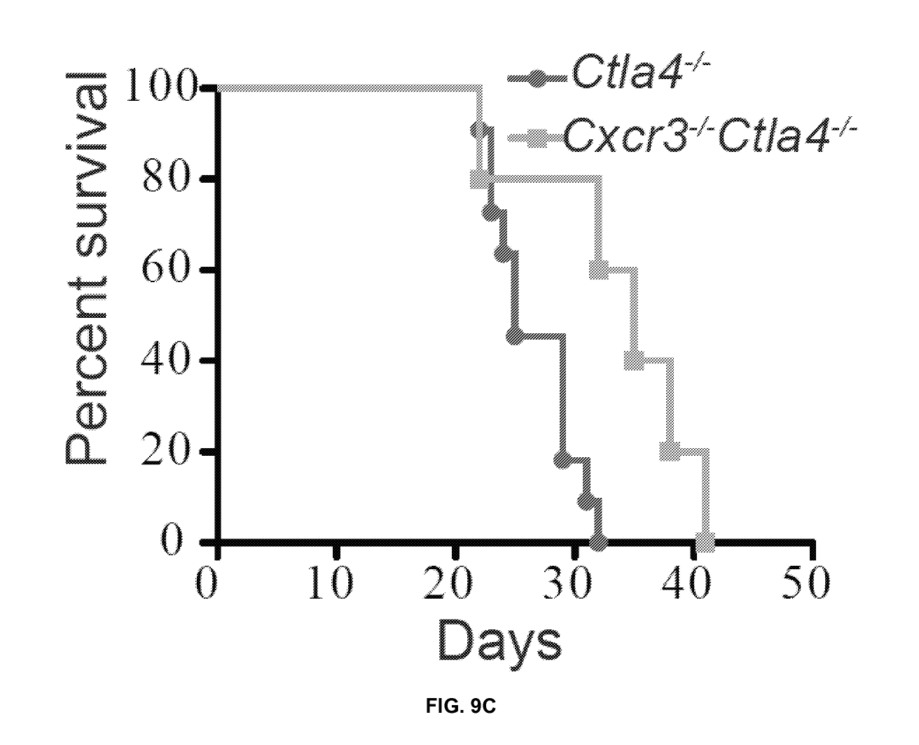

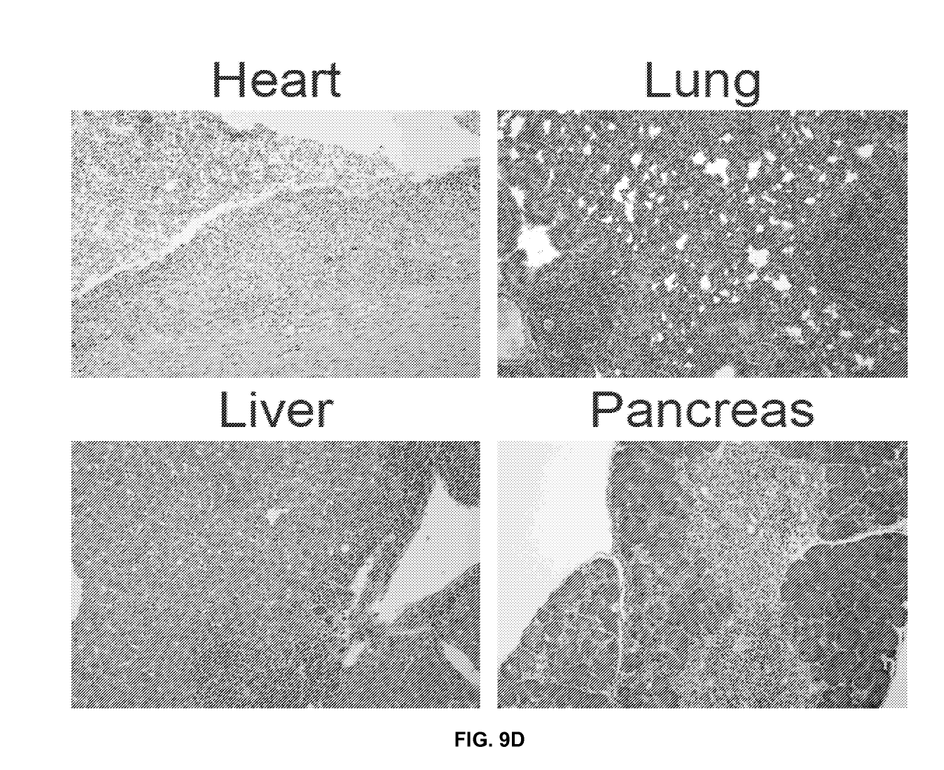

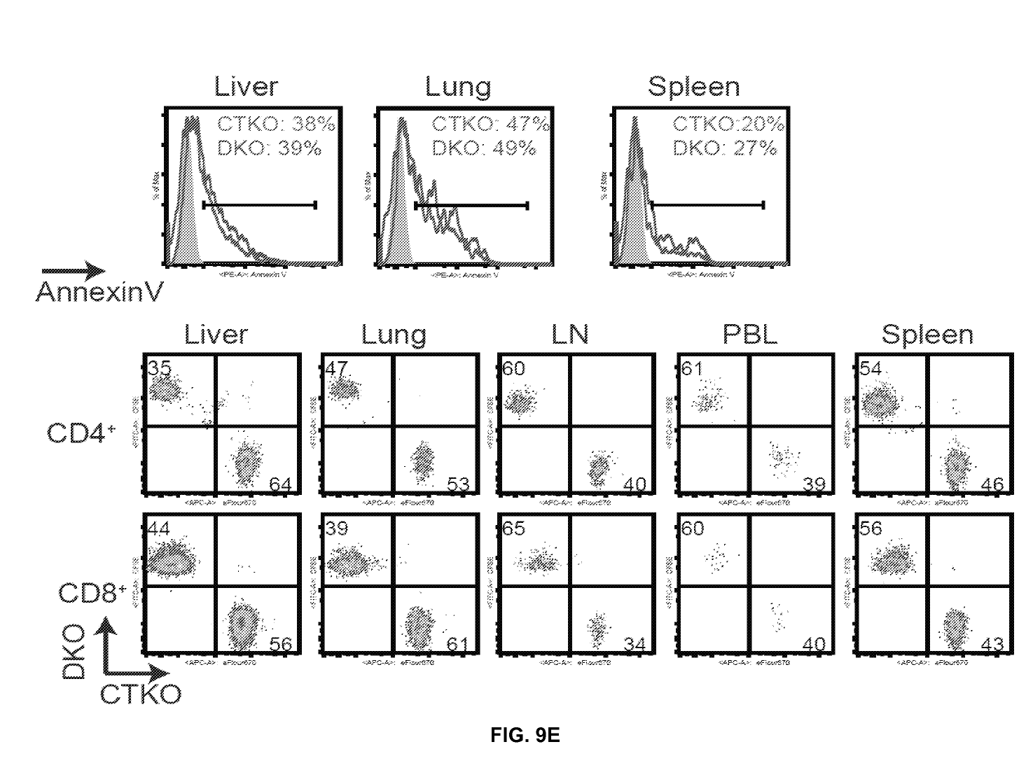

FIG. 9: Tissue homing defects of DKO T cells cannot be solely accounted for by alterations in CXCR3 expression and function.
A. Expression of the chemokine receptor, CXCR3, on activated CD4⁺CD25^{neg}CD44^{hi} Tconv cells in peripheral blood and LNs of 8 weeks old WT, Itk^{-/-} and DKO mice and 3 weeks old Ctla4^{-/-} mice. B. Frequency of migration of CD4⁺ T cells from Ctla4^{-/-} and DKO mice to indicated concentrations of the CXCR3 ligand CXCL-11 in vitro. C. Kaplan-Meier survival curves of Ctla4^{-/-} (n=10) and Cxcr3^{-/-} Ctla4^{-/-} (n=5) mice show an approximate 10-day extension in the lifespan of the latter. D. Representative H&E stained sections from indicated organs of 3-4 weeks old Cxcr3^{-/-} Ctla4^{-/-} mice show massive infiltration into non-lymphoid tissue by Cxcr3^{-/-} Ctla4^{-/-} T cells, indicating that decreased CXCR3 expression alone cannot account for the lack of infiltration and autoimmunity in Itk^{-/-} Ctla4^{-/-} DKQ mice. E. (Top) Representative histogram overlays showing similar AnnexinV staining on Ctla4^{-/-} (blue) and DKO (red) CD4⁺ T cells isolated from the liver, lungs and spleen of Rag1^{-/-} mice that had received a mix of eFluor670-labeled Ctla4^{-/-} T cells and CFSE-labeled DKO T cells 6-8 hours prior to analysis. Gray shaded histograms are the unstained controls. (Bottom) Representative dot plots show distribution of eFluor670-labeled Ctla4^{-/-} and CFSE-labeled DKO CD8⁺ and CD4⁺ T cells in the liver, lungs, lymph nodes (LN), peripheral blood and spleen of Rag1^{-/-} mice that had received a 1:1.5 mix of Ctla4^{-/-} and DKO T cells 6-8 hours prior to analysis.

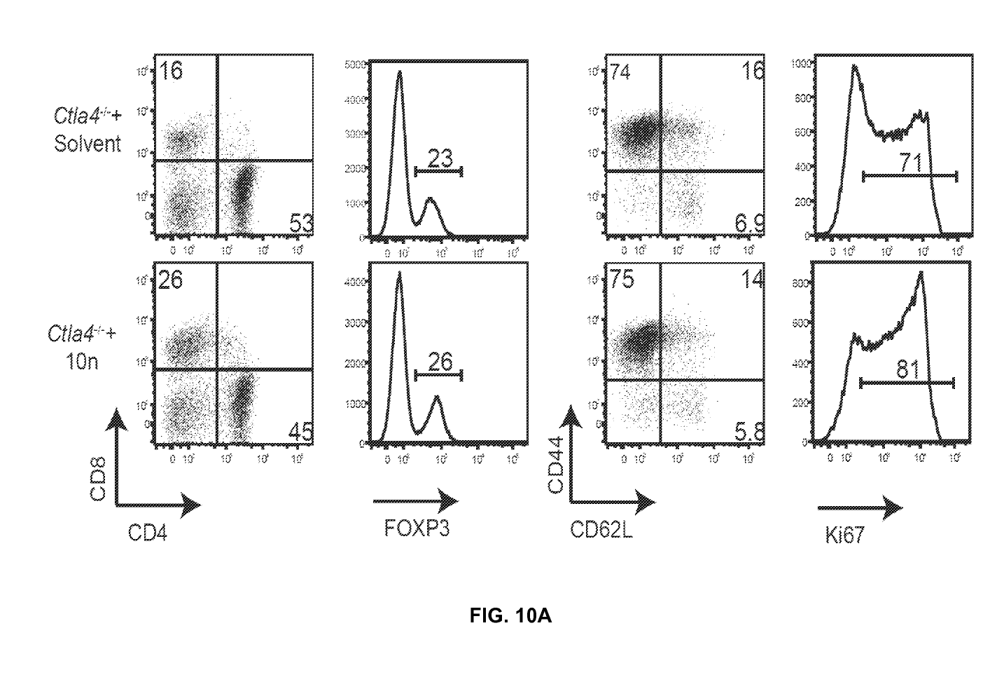

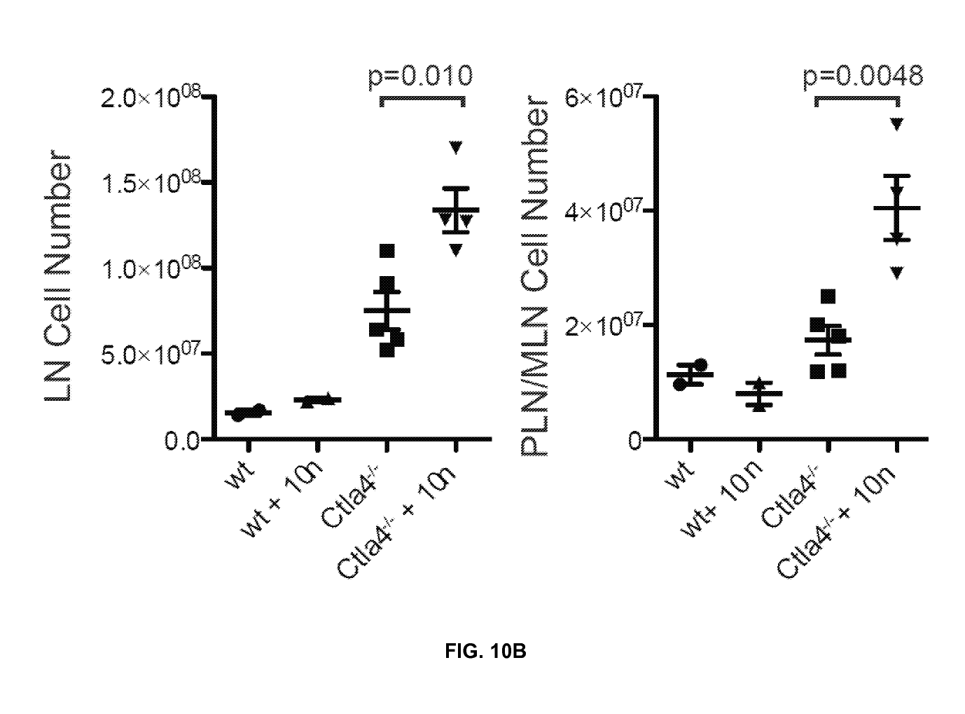

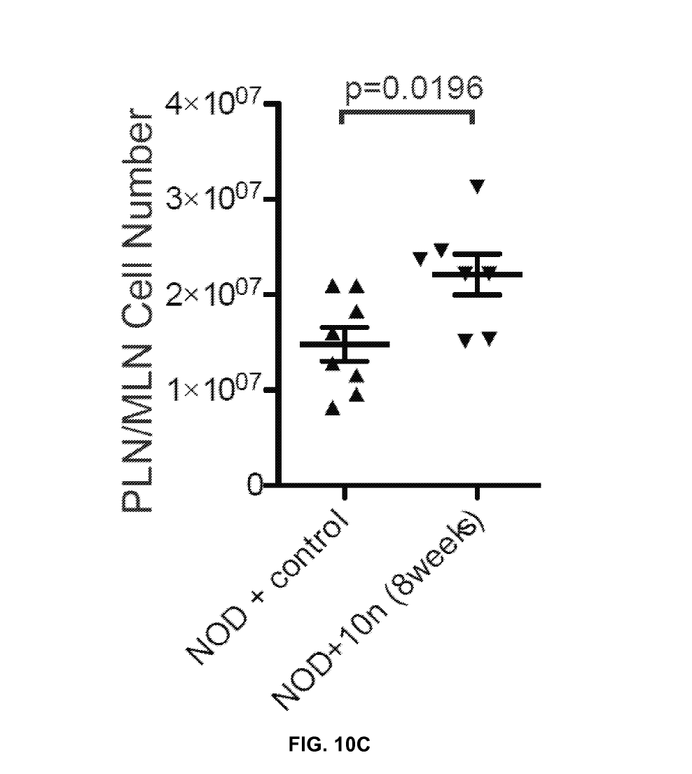

FIG. 10: ITK inhibitor treatment does not alter activation of auto-reactive T cells but affects trafficking in vivo.
A. Frequency of CD4⁺, CD8⁺ and CD4⁺FOXP3⁺ T cells in peripheral LNs of Ctla4^{-/-} mice treated with solvent or ITK inhibitor at 3.5 weeks of age. CD4⁺FOXP3^{neg} Tconv cells were further analyzed for the expression of activation markers, CD44 and

CD62L, and proliferation marker, Ki67. Data are representative of two independent experiments with two mice in each group. B. Cellularity of peripheral LNs (Left) and pooled mesenteric and pancreatic LNs (Right) of 3.5 weeks old WT and Ctla4^{-/-} mice treated with the ITK inhibitor 10n. Data are pooled from two experiments with at least two mice in each group. C. Cellularity of pooled mesenteric and pancreatic LNs from 8 weeks old female NOD mice treated with the ITK inhibitor 10n or solvent alone. Data are pooled from two experiments with at least three mice in each group.

### DETAILED DESCRIPTION

CD28 is the primary costimulatory molecule for naive conventional CD4⁺ T (Tconv) cell activation (Bour-Jordan et al., Immunol Rev. 241, 180-205, 2011). CD28 binding to B7 ligands on the antigen presenting cell (APC) transduces signals that increase the duration and the magnitude of the T cell response(Harding et al., Nature 356, 607-609, 1992), induction of anti-apoptotic proteins(Boise et al., Immunity 3, 87-98, 1995), enhanced glucose metabolism (Frauwirth et al., Immunity 16, 769-777, 2002) and acquisition of proper migratory properties (Marelli-Berg et al., Trends Immunol. 28, 267-273, 2007). CD28 costimulation activates integrin-mediated adhesion of T cells (Shimizu et al., J Exp Med. 175, 577-582, 1992) and promotes actin polymerization by activating VAV1 and Cdc42 (Michel et al., J Immunol. 165, 3820-3829, 2000; Salazar-Fontana, et al., J Immunol. 171, 2225-2232, 2003). CD28 signals are also necessary for the generation and maintenance of CD4⁺FOXP3⁺ regulatory T (Treg) cells (Salomon et al., Immunity 12, 431-440, 2000).

The effects of co-stimulatory blockade in autoimmune disease progression in mice are complex. Cd28^{-/-} mice have impaired delayed-type hypersensitivity responses (Kondo et al., J Immunol. 157, 4822-4829, 1996) and fail to develop Experimental

Autoimmune Encephalitis (EAE) (Girvin et al., J Immunol. 164, 136-143, 2000, where B7 is required for the initiation of autoreactive T cell activation as well as their survival in brain parenchyma (Chang et al., J Exp Med. 190, 733-740, 1999). In the non-obese diabetic (NOD) mouse model of spontaneous Type 1 diabetes (T1D), the loss of CD28 exacerbates disease(Lenschow et al., Immunity 5, 285-293, 1996), probably due to a decreased frequency of FOXP3⁺ Treg cells that results in a loss of tolerance to pancreatic antigens (Salomon et al., Immunity 12, 431-440, 2000). However, NOD mice treated with CTLA4Ig (Abatacept), a fusion protein that binds to and sequesters B7, are protected from diabetes (Lenschow et al., J Exp Med. 181, 1145-1155, 1995). Further, CD28 costimulation in neonatal NOD mice promotes the production of IL-4 by islet-infiltrating T cells, which moderates disease, an outcome that is not seen in adult-treated NOD mice (Arreaza et al., J Clin Invest. 100, 2243-2253, 1997). The interpretation of these studies is complicated by the function of the CD28 antagonist, CTLA-4, a co-inhibitor that also binds B7, but with a much higher affinity than CD28 (Chambers et al., Ann Rev Immunol. 19, 565-594, 2001; Greenwald et al., Ann Rev Immunol. 23, 515-548, 2005).

As a counter balance to CD28, the primary function of CTLA-4 is to maintain peripheral T cell tolerance (Chambers et al., Ann Rev Immunol. 19, 565-594, 2001), and polymorphisms in Ctla4 have been associated with human autoimmune disease susceptibility (Gough et al., Immunol Rev. 204, 102-115, 2005). Ctla4^{-/-} mice suffer from a fatal lymphoproliferative disorder characterized by overt CD28-dependent CD4⁺ T cell activation and damaging infiltration of self-antigen reactive T cells into tissues (Chambers et al., Immunity 7, 885-895, 1997; Ise et al., Nat Immunol. 11, 129-135, 2010). This loss in self-tolerance is initiated by the inability of CTLA-4-deficient FOXP3⁺ Treg cells to maintain immune homeostasis (Ise et al., Nat Immunol. 11, 129-135, 2010; Wing et al., Science 322, 271-275, 2008; Friedline et al., J Exp Med. 206, 421-434, 2009; Jain et al., Proc Natl Acad Sci USA 107, 1524-8., 2010), resulting in hyper-stimulatory APCs (Wing et al., Science 322, 271-275, 2008; Friedline et al., J Exp Med. 206, 421-434, 2009). CTLA-4 also has Tconv cell intrinsic functions and can regulate trafficking of self-reactive T cell to tissues (Ise et al., Nat Immunol. 11, 129-135, 2010; Jain et al., Proc Natl Acad Sci USA 107, 1524-8., 2010). Over-expression of a mutant CTLA-4 protein containing only the B7-binding domain protects Ctla4^{-/-} mice from organ infiltration by activated T cells (Masteller et al., J Immunol. 164, 5319-5327, 2000). These results suggest that modulation of CD28 signals by competitive sequestration of B7 ligands can protect mice from tissue infiltration by autoreactive T cells.

The biochemical pathway downstream of CD28 specifically controlling T cell migration is beginning to be elucidated. Studies with TCR transgenic (Tg) T cells have suggested the involvement of CD28-induced PI3Kinase (PI3K) activation in the trafficking of effector and memory T cells to target tissues (Okkenhaug et al., Nat Immunol. 2, 325-332, 2001; Mirenda et al., Blood 109, 2968-2977, 2007). The IL-2 inducible Tec kinase ITK is an enzyme that is recruited to both the TCR and CD28 upon stimulation in a PI3K-dependent manner (Michel et al., Immunity 15, 935-945, 2001). Phosphorylated ITK activates PLC-γ1, leading to calcium (Ca²⁺) mobilization and the activation of PKC and RAS/ERK signaling pathway, and actin polarization to the site of TCR stimulation via the Rac and Cdc42 pathways (Berg et al., Ann Rev Immunol. 23, 549-600, 2005). However, ITK appears dispensable for activated T cell localization to target tissues in infectious and adjuvant treated in vivo settings (Fowell et al., Immunity 11, 399-409, 1999). Whether CD28-ITK signaling is necessary for the migration of spontaneously arising self-reactive T cells in vivo is not known. While Cd28^{-/-} mice do not exhibit impairments in activated T cell migration to tissues upon pathogen infection (Greenwald et al., Ann Rev Immunol. 23, 515-548, 2005), we show here that CD28-B7 interactions regulate self-reactive T cell migration in tissues. We generated Itk^{-/-}Ctla4^{-/-} mice to determine if ITK is essential for autoreactive T cell migration. While the characteristic CD28-dependent rampant activation and lymphoproliferation of CTLA-4 deficient CD4⁺ T cells are present in these mice, loss of ITK altered the B7-dependent migratory behavior of activated self-reactive T cells. This resulted in strikingly reduced numbers of pathogenic T cells in non-lymphoid organs, which dramatically extended the lifespan of Ctla4-deficient mice to over a year. Importantly, small molecule inhibitors of ITK could moderate autoimmunity in Ctla4^{-/-} mice, and significantly diminished T cell infiltration and destruction of islet cells in T1D models. Our results implicate a biased dependence on CD28-B7 signaling involving ITK in the trafficking of self-reactive T cells, and provide proof of principle that targeting ITK may be beneficial for the treatment of T cell-mediated human organ-specific autoimmune diseases.

CTLA-4 and CD28 are critical for regulating autoimmunity such as T1D in mice and humans, but are often dispensable for responses against foreign pathogens (Lenschow et al., Immunity 5, 285-293, 1996; Friedline et al., J Exp Med. 206, 421-434, 2009; Bachmann et al., J Immunol. 160, 95-100, 1998; Shahinian et al., Science 261, 521-652, 1993). Most in vivo studies support the role of ligand competition as the primary mode of CTLA-4 inhibition of CD28 signals (Masteller et al., J Immunol. 164, 5319-5327, 2000; Collins et al., Immunity 17, 201-210, 2002; Yokosuka et al., Immunity 33, 326-339, 2010; Puccetti et al., Nat Rev Immunol. 7, 817-823, 2007; Qureshi et al., Science 332, 600-603, 2011). We show here that CD28-B7 interactions and ITK regulate the trafficking of self-reactive T cells to tissues. Given the established biochemical connection between CD28 and ITK, these data support the model that one Tconv cell-intrinsic function of CTLA-4 is to modulate the CD28-ITK pathway controlling self-reactive T cell motility in tissues.

CD28 signaling is necessary for the activation of self-reactive Ctla4^{-/-} T cells in vivo (Mandelbrot et al., J Clin Invest. 107, 881-887, 2001). While altering specific downstream mediators of CD28 in Ctla4^{-/-} mice was predicted to reveal pathways that contribute to the pathogenesis of T cell mediated autoimmunity, the challenge was to identify components that have biased function in specific facets of CD28-mediated disease pathogenesis. ITK was an ideal candidate, since the loss-of-function of ITK does not completely abolish TCR-CD28 signaling, but primarily impairs the PI3K-PLCγ1 pathway (Berg et al., Ann Rev Immunol. 23, 549-600, 2005). Consequently, naïve DKO CD4⁺ T cells respond to their cognate antigens comparably to WT T cells, and DKO mice are replete with activated T cells; these findings established that the CD28 costimulatory signals necessary for the initial activation of Ctla4^{-/-} T cells are intact in the absence of ITK. Further, DKO mice are effective in pathogen clearance, again confirming that T cell activation and differentiation are not globally impaired. Hence, the inability of activated DKO T cells to enter tissues and to cause fatal autoimmunity revealed a specific function of ITK in self-reactive T cell trafficking. In addition to CD28 signaling, ITK has been implicated in integrin and chemokine receptor signaling (Woods et al., EMBO J. 20, 1232-1244, 2001; Takesono et al., Curr Biol. 14, 917-922, 2004; Sahu et al., J Immunol. 180, 3833-3838, 2008); however, the major consequence of this in Itk^{-/-} mice is impaired lymphocyte trafficking to the LNs. In models of infectious diseases, ITK-deficient T cells did not show significant alterations in their migratory property (Bachmann et al., J Virol. 71, 7253-7257, 1997; Au-Yeung et al., J Immunol. 176, 3895-3899, 2006). DKO T cells were impaired in responding to CXCR3 ligands in vitro; however despite this, Cxcr3^{-/-}Ctla4^{-/-} mice had extensive T cell infiltrates into tissues and a short life span, indicating that possible defects in CXCR3 signaling due to ITK-deficiency cannot fully account for the lack of T cell infiltrates in DKO mice.

Lastly, we showed that a small molecule ITK inhibitor prevents the migration of activated diabetogenic BDC2.5 CD4⁺ T cells into the pancreas. These findings indicate that the requirement for ITK in autoimmune pathogenesis first identified with Ctla4^{-/-} T cells is not unique and is likely to be relevant for self-reactive T cells in various human organ specific autoimmune disorders. Costimulatory blockade is a major therapeutic strategy for autoimmune diseases such as rheumatoid arthritis and T1D. Abatacept, a CD28 antagonist, has achieved moderate success in T1D patients, leading to the preservation of beta-cell mass for over two years (Orban et al., Lancet 378, 412-419, 2011). However, this approach ultimately fails due to the increased antigen sensitivity of autoreactive T cells, and their relative independence from costimulation for their activation. Another major drawback is the failure to specifically target auto-reactive T cells as B7 blockade can interfere with Treg cell function (Salomon et al., Immunity 12, 431-440, 2000). In contrast, focused approaches aimed at regulating self-reactive T cell migration to organs to limit immune pathology are beneficial in treating severe autoimmunity (Bauer et al., Proc Natl Acad Sci USA 106, 1920-1925, 2009). ITK has been shown to have a similar functional repertoire in human T cells as in mice, including the ability to modulate T cell actin polarization (Readinger et al., Proc Natl Acad Sci USA 105, 6684-6689, 2008; Dombroski et al., J Immunol. 174, 1385-1392, 2005; Guo et al., Mol Pharmacol. 82, 938-947, 2012). Our data indicate that ITK inhibitors can become an alternate strategy to treat diverse human T cell mediated organ-specific autoimmune diseases, while allowing pathogen-driven immune responses to occur.

The methods described herein are based, at least in part, on the discovery that ITK and CD28 signals regulate autoreactive T cell trafficking and that organ-specific T cell mediated autoimmune diseases, e.g., Type 1 diabetes, Hashimoto's thyroiditis, multiple sclerosis, non-infectious uveitis, Sjögren's syndrome, primary biliary cirrhosis, autoimmune hepatitis, and ankylosing spondylitis, can be treated by inhibiting ITK. Accordingly, a therapeutic strategy provided herein involves identifying a subject in need of treatment for an organ-specific T cell mediated autoimmune disease and inhibiting ITK by administering to the subject a therapeutically effective amount of an ITK inhibitor, e.g., BMS509744, ibrutinib, 10n, 2-amino-5-(thioaryl)thiazole, 5 aminomethylbenzimidazole, 2-amino-5-[(thiomethyparyl]thiazole, and biaryl thiophene, thereby treating the subject with the organ-specific T cell mediated autoimmune disease. In some embodiments, the methods described herein include administering abatacept, secukinumab, or infliximab to the subject. The ITK inhibitor can be administered to the subject, e.g., a mammal, e.g., a human, orally, intravenously, or by injection. Inhibiting ITK function with BMS509744, ibrutinib, 10n, 2-amino-5-(thioaryl)thiazole, 5 aminomethylbenzimidazole, 2-amino-5-[(thiomethyl)aryl]thiazole, or biaryl thiophene can be particularly helpful as the method can inhibit migration of autoreactive T cells into a target tissue, and migration of alloreactive T cells is unaffected.

Activation of self-reactive T cells and their trafficking to target tissues leads to autoimmune organ destruction. Mice deficient for the inhibitory receptor CTLA-4 develop fatal autoimmunity characterized by massive lymphocytic invasion into non-lymphoid tissues. The present disclosure demonstrates that the B7-CD28 costimulatory pathway regulates the trafficking of self-reactive Ctla4^{-/-} T cells to tissues. Co-ablation of the CD28-activated Tec family kinase ITK does not block spontaneous T cell activation, but instead causes self-reactive Ctla4^{-/-} T cells to accumulate in secondary lymphoid organs. Despite a fulminant autoimmune process in the lymphoid compartment, Itk^{-/-}Ctla4^{-/-} mice are otherwise healthy and exhibit a long lifespan. ITK licenses autoreactive T cells to enter tissues to mount destructive immune responses. Importantly, ITK inhibitors mimic the null mutant phenotype and also prevent pancreatic islet infiltration by diabetogenic T cells in mouse models of Type 1 diabetes, highlighting their potential utility for the treatment of human autoimmune disorders, including Type 1 diabetes, Hashimoto's thyroiditis, multiple sclerosis, non-infectious uveitis, Sjögren's syndrome, primary biliary cirrhosis, autoimmune hepatitis, and ankylosing spondylitis.

### ITK Inhibitors

BMS509744

BMS509744 is a cell-permeable aminothioaryl-thiazolo compound that potently inhibits ITK kinase activity in an ATP-competitive manner by stabilizing ITK activation loop in a substrate-blocking, inactive conformation, inhibiting Fyn, IR, Lck, Btk only at high concentrations and exhibiting little or no activity against 14 other kinases. BMS509744 is commercially available from Boehringer Ingelheim Pharmaceuticals, Inc. and EMD Millipore (Cat. No. 419820-5MG).

Ibrutinib

Ibrutinib is an experimental drug candidate for the treatment of various types of cancer. It is a selective and covalent inhibitor of the enzyme Bruton's tyrosine kinase (BTK).

10n

Originally discovered by Boehringer Ingelheim Pharmaceuticals, Inc., 10n is a lipophilic 5-aminobenzimidazole analog ITK inhibitor in which the benzamide linker has been replaced with (1S)-1,2,2-Trimethylpropyl, to block the generation of toxic metabolites in vivo from the amide bond hydrolysis. 5-aminobenzimidazole binds to the kinase specificity pocket of ITK and 10n has negligible reactivity to insulin receptor tyrosine kinase, which is structurally similar to ITK.

Other ITK inhibitors such as 2-amino-5-(thioaryl)thiazoles, 5-aminomethylbenzimidazoles, and 2-amino-5-[(thiomethyparyl]thiazoles are available from Bristol-Myers Squibb), and biaryl thiophenes are available from Boehringer Ingelheim.

### Treatable Subjects

A subject who can be treated using the methods described herein can be identified on the basis that they have Type 1 diabetes, Hashimoto's thyroiditis, multiple sclerosis, non-infectious uveitis, Sjögren's syndrome, primary biliary cirrhosis, autoimmune hepatitis, or ankylosing spondylitis, are suspected to have Type 1 diabetes, Hashimoto's thyroiditis, multiple sclerosis, non-infectious uveitis, Sjögren's syndrome, primary biliary cirrhosis, autoimmune hepatitis, or ankylosing spondylitis, and/or are at risk of having Type 1 diabetes, Hashimoto's thyroiditis, multiple sclerosis, non-infectious uveitis, Sjögren's syndrome, primary biliary cirrhosis, autoimmune hepatitis, or ankylosing spondylitis. It is well within the skills of an ordinary practitioner to recognize a subject that has, is suspected to have, or is at risk of having Type 1 diabetes, Hashimoto's thyroiditis, multiple sclerosis, non-infectious uveitis, Sjögren's syndrome, primary biliary cirrhosis, autoimmune hepatitis, or ankylosing spondylitis.

### Type 1 Diabetes

Type 1 diabetes is a lifelong (chronic) disease in which there is a high level of glucose in the blood. Type 1 diabetes can occur at any age. It is most often diagnosed in children, adolescents, or young adults. Insulin is a hormone produced in the pancreas by special cells, called beta cells. The pancreas is behind the stomach. Insulin is needed to move blood glucose into cells. There, it is stored and later used for energy. In Type 1 diabetes, beta cells produce little or no insulin. Without enough insulin, glucose builds up in the bloodstream instead of going into the cells. The body is unable to use this glucose for energy. This leads to the symptoms of Type 1 diabetes. The exact cause of Type 1 diabetes is unknown. Most likely it is an autoimmune disorder. This is a condition that occurs when the immune system mistakenly attacks and destroys healthy body tissue. With Type 1 diabetes, an infection or another trigger causes the body to mistakenly attack the islet cells in the pancreas that make insulin. Type 1 diabetes can be passed down through families.

Symptoms of Type 1 diabetes include, for example, being very thirsty, feeling hungry, fatigue, blurry eyesight, feeling numbness or feeling tingling in the feet, losing weight without trying, urinating more often, deep, rapid breathing, dry skin and mouth, flushed face, fruity breath odor, nausea or vomiting, inability to keep down fluids, and stomach pain.

### Hashimoto's Thyroiditis

Hashimoto's thyroiditis, also known as chronic lymphocytic thyroiditis and autoimmune thyroiditis, is inflammation of the thyroid gland that often results in reduced thyroid function (hypothyroidism). Hashimoto's thyroiditis is a common thyroid gland disorder. It can occur at any age, but is most often seen in middle-aged women. It is caused by a reaction of the immune system against the thyroid gland. The disease begins slowly. It may take months or even years for the condition to be detected. Hashimoto's thyroiditis is most common in women and in people with a family history of thyroid disease. It affects between 0.1% and 5% of all adults in Western countries. Hashimoto's thyroiditis may, in rare cases, be related to other endocrine disorders caused by the immune system. Hashimoto's thyroiditis can occur with adrenal insufficiency and Type 1 diabetes. In these cases, the condition is called type 2 polyglandular autoimmune syndrome (PGA II). Less commonly, Hashimoto's thyroiditis occurs as part of a condition called type 1 polyglandular autoimmune syndrome (PGA I), along with adrenal insufficiency, fungal infections of the mouth and nails, and hypoparathyroidism.

Symptoms of Hashimoto's thyroiditis include, for example, constipation, difficulty concentrating or thinking, dry skin, enlarged neck or presence of goiter, fatigue, hair loss, heavy and irregular periods, intolerance to cold, mild weight gain, small or shrunken thyroid gland (late in the disease), joint stiffness, unintentional weight gain, and swelling of the face.

### Multiple Sclerosis

Multiple sclerosis (MS) is an autoimmune disease that affects the central nervous system. MS affects women more than men. The disorder is most commonly diagnosed between ages 20 and 40, but can be seen at any age. MS is caused by damage to the myelin sheath, the protective covering that surrounds nerve cells. When this nerve covering is damaged, nerve signals slow down or stop. The nerve damage is caused by inflammation. Inflammation occurs when the body's own immune cells attack the nervous system. This can occur along any area of the brain, optic nerve, and spinal cord. It is unknown what exactly causes this to happen. The most common thought is that a virus or gene defect, or both, is to blame. Environmental factors may play a role, and one is more likely to get MS if there is a family history of MS or if one lives in a part of the world where MS is more common.

A fever, hot bath, sun exposure, or stress can trigger or worsen attacks. It is common for the disease to return. However, the disease may continue to get worse without periods of remission. Because nerves in any part of the brain or spinal cord may be damaged, patients with MS can have symptoms in many parts of the body. Symptoms of MS include, for example, fatigue, loss of balance, muscle spasms, numbness or abnormal sensation in any area, problems moving arms or legs, problems walking, problems with coordination and making small movements, tremor in one or more arms or legs, weakness in one or more arms or legs, constipation and stool leakage, difficulty beginning to urinate, frequent need to urinate, strong urge to urinate, incontinence, double vision, eye discomfort, uncontrollable rapid eye movements, vision loss, facial pain, painful muscle spasms, tingling, crawling, or burning feeling in the arms and legs, decreased attention span, poor judgment, and memory loss, difficulty reasoning and solving problems, depression or feelings of sadness, dizziness and balance problems, hearing loss, erectile dysfunction, problems with vaginal lubrication, slurred or difficult-to-understand speech, and trouble chewing and swallowing.

### Non-Infectious Uveitis

Uveitis is broadly defined as an inflammation of the uvea. The uvea consists of the middle, pigmented, vascular structures of the eye and includes the iris, ciliary body, and choroid. Uveitis requires an urgent referral and thorough examination by an ophthalmologist or optometrist and urgent treatment to control the inflammation. The cause of non-infectious uveitis is unknown, but there are some strong genetic factors that predispose disease onset, including HLA-B27 and the PTPN22 genotype.

Symptoms of non-infectious uveitis include, for example, redness of the eye, blurred vision, photophobia or sensitivity to light, eye pain, floaters, which are dark spots that float in the visual field, headaches, and busacca nodules. Signs of anterior uveitis include dilated ciliary vessels, presence of cells and flare in the anterior chamber, and keratic precipitates on the posterior surface of the cornea. In severe inflammation there may be evidence of a hypopyon. Old episodes of uveitis are identified by pigment deposits on lens, keratic precipitates, and festooned pupil on dilation of pupil.

### Sjögren's Syndrome

Sjögren's syndrome is an autoimmune disorder in which the glands that produce tears and saliva are destroyed, leading to dry mouth and dry eyes. The condition may affect other parts of the body, including the kidneys and lungs. The cause of Sjögren's syndrome is unknown. It is an autoimmune disorder that occurs most often in women ages 40 to 50. It is rare in children. Dry eyes and dry mouth are the most common symptoms of this syndrome. Other symptoms can include, for example, itching eyes, feeling that something is in the eye, difficulty swallowing or eating, loss of sense of taste, problems speaking, thick or stringy saliva, mouth sores or pain, hoarseness, fatigue, fever, change in the color of hands or feet, joint pain or joint swelling, and swollen glands.

### Primary Biliary Cirrhosis

Primary biliary cirrhosis is irritation and inflammation of the bile ducts of the liver, which blocks the flow of bile. This obstruction damages liver cells and leads to scarring called cirrhosis. The cause of inflamed bile ducts in the liver is not known. However, primary biliary cirrhosis is an autoimmune disorder that more commonly affects middle-aged women. More than half of patients have no symptoms at the time of diagnosis. Symptoms usually come on gradually and may include, for example, abdominal pain, an enlarged liver, fatigue, fatty deposits under the skin, fatty stools, itching, jaundice, and soft yellow spots on the eyelid.

### Autoimmune Hepatitis

Autoimmune hepatitis is inflammation of the liver that occurs when immune cells mistake the liver's normal cells for harmful invaders and attack them. This form of hepatitis is an autoimmune disease resulting in an immune response that destroys normal body tissues. Autoimmune hepatitis sometimes occurs in relatives of people with autoimmune diseases, which suggests that there may be a genetic cause. This disease is most common in young girls and women. Symptoms of autoimmune hepatitis include, for example, abdominal distention, dark urine, fatigue, general discomfort, uneasiness, or ill feeling (malaise), itching, loss of appetite, nausea or vomiting, pale or clay-colored stools, and an absence of menstruation (amenorrhea).

### Ankylosing Spondylitis

Ankylosing spondylitis is a long-term type of arthritis that affects the bones and joints at the base of the spine where it connects with the pelvis. These joints become swollen and inflamed and over time, the affected spinal bones join together. The disease starts with low back pain that comes and goes. Low back pain is present most of the time as the condition progresses. Pain and stiffness are worse at night, in the morning, or when less active. The pain often gets better with activity or exercise. Back pain may begin in the sacroiliac joints (between the pelvis and spine). Over time, it may involve all or part of the spine. Flexibility may be lost in the lower spine. The condition may affect the joints between the ribs so that the chest cannot be expanded fully. Fatigue is also a common symptom. Other symptoms can include swelling of the eye, heel pain, hip pain and stiffness, swelling and pain in the joints of the shoulders, knees, and ankles, loss of appetite, slight fever, and weight loss.

### Methods of Treatment

The methods described herein include methods for the treatment of organ-specific T cell mediated autoimmune diseases, e.g., Type 1 diabetes, Hashimoto's thyroiditis, multiple sclerosis, non-infectious uveitis, Sjögren's syndrome, primary biliary cirrhosis, autoimmune hepatitis, and ankylosing spondylitis. In some embodiments, the method inhibits migration of autoreactive T cells into a target tissue. In one embodiment, the method does not affect migration of alloreactive T cells. Generally, the methods include identifying a subject in need of treatment for an organ-specific T cell mediated autoimmune disease; and administering to the subject a therapeutically effective amount of an ITK inhibitor, wherein the ITK inhibitor is selected from the group consisting of BMS509744, ibrutinib, 10n, 2-amino-5-(thioaryl)thiazole, 5-aminomethylbenzimidazole, 2-amino-5-[(thiomethyparyl]thiazole, and biaryl thiophene, thereby treating the subject with the organ-specific T cell mediated autoimmune disease.

As used in this context, to “treat” means to ameliorate at least one symptom of an organ-specific T cell mediated autoimmune disease. In some embodiments, the subjects treated by the methods described herein have Type 1 diabetes, i.e., are diabetic. A person who is diabetic has one or more of a Fasting Plasma Glucose Test result of 126 mg/dL or more; a 2-Hour Plasma Glucose Result in an Oral Glucose Tolerance Test of 200 mg/dL or more; and blood glucose level of 200 mg/dL or above. In some embodiments, the subjects treated by the methods described herein are being treated for diabetes, e.g., have been prescribed or are taking insulin, meglitinides, biguanides, thiazolidinediones, or alpha-glucosidase inhibitors.

In some embodiments the subjects are pre-diabetic, e.g., they have impaired glucose tolerance or impaired fasting glucose, e.g., as determined by standard clinical methods such as the intravenous glucose tolerance test (IVGTT) or oral glucose tolerance test (OGTT), e.g., a value of 7.8-11.0 mmol/L two hours after a 75 g glucose drink for impaired glucose tolerance, or a fasting glucose level (e.g., before breakfast) of 6.1-6.9 mmol/L.

In some embodiments, the methods described herein include selecting subjects who have diabetes or pre-diabetes. In some embodiments, the following table is used to identify and/or select subjects who are diabetic or have pre-diabetes, i.e., impaired glucose tolerance and/or impaired fasting glucose.

Fasting Blood Glucose

| | |
|---|---|
| From 70 to 99 mg/dL | Normal fasting glucose |
| (3.9 to 5.5 mmol/L) | |
| From 100 to 125 mg/dL | Impaired fasting glucose |
| (5.6 to 6.9 mmol/L) | (pre-diabetes) |
| 126 mg/dL (7.0 mmol/L) | Diabetes |
| and above on more than | |
| one testing occasion | |

Oral Glucose Tolerance Test (OGTT)
[except pregnancy]
(2 hours after a 75-gram glucose drink)

| | |
|---|---|
| Less than 140 mg/dL | Normal glucose tolerance |
| (7.8 mmol/L) | |
| From 140 to 200 mg/dL | Impaired glucose tolerance |
| (7.8 to 11.1 mmol/L) | (pre-diabetes) |
| Over 200 mg/dL (11.1 mmol/L) | Diabetes |
| on more than one testing occasion | |

### Dosage

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a therapeutic compound (i.e., an effective dosage) depends on the therapeutic compounds selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

Dosage, toxicity and therapeutic efficacy of the therapeutic compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. ITK inhibitors that exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

### Methods of Administration

The methods described herein include the use of pharmaceutical compositions, which include compounds that target ITK as active ingredients. Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions, e.g., anti-inflammatory drugs as are known in the art.

Pharmaceutical compositions are typically formulated to be compatible with its intended route of administration. Examples of routes of administration include parenteral, e.g., intravenous, intradermal, subcutaneous, oral, nasal, transdermal (topical), transmucosal, and rectal administration. The route of administration can be selected by one of skill on the art and will depend on the nature of the active compound.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, N.Y.). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressured container or dispenser that contains a suitable propellant, e.g., a gas such as carbon dioxide, or a nebulizer. Such methods include those described in U.S. Pat. No. 6,468,798.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The pharmaceutical compositions can also be prepared in the form of suppositories (e.g., with conventional suppository bases such as cocoa butter and other glycerides) or retention enemas for rectal delivery.

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration.

### EXAMPLES

The invention is further described in the following examples, which do not limit the scope of the invention described in the claims.

### Methods

Mice

Itk^{-/-} mice were crossed with Ctla4^{-/-} mice to generate Itk^{-/-}Ctla4^{-/-} (DKO) mice on the C57/BL6 background. Female NOD, NOD/Scid mice, Cxcr3^{-/-} and Cd80^{-/-}Cd86^{-/-} (B7^{-/-}) mice were purchased from Jackson Laboratories (Bar Harbor, Me.). BDC2.5/NOD mice were bred in our animal facility. Cd80^{-/-}Cd86^{-/-}Rag1^{-/-} and 5C.C7Itk^{-/-}Ctla4^{-/-}Rag1^{-/-} mice were generated in our colony. Rag1^{-/-}H-2Aα^{-/-}Cd74^{-/-} (no I-A, E; MHC Class II^{-/-}) mice were provided by Dr. Huseby. All experiments were approved by the University of Massachusetts Medical School Institutional Animal Care and Use Committee.

Reagents, Antibodies, Flow Cytometry and Histology

Pertussis toxin was from List Biological Laboratories, Campbell CA; ITK inhibitors, BMS507944 and 10n, were synthesized at the NIH's Chemical Genomics Centre and dissolved in 70% (20% w/v) 2-hydroxypropyl-B-cyclodextrin in water and 30% PEG300 (EMD), CD28 superagonist antibody (SACD28, clone D665) was a gift from Dr. T Hunig and SIP was from R&D Systems. Most antibodies used in these experiments were purchased from BD Bioscience (San Jose, Calif.) and eBioscience (San Diego, Calif.). All data were acquired on the LSRII flow cytometer (BD), and analyzed using FlowJo software (Treestar, Calif.). Peripheral T cell subsets were sorted to greater than 95% purity using a MoFlo (Cytomation) cell sorter. CD4⁺ T cells were enriched from SLOs by depleting CD8⁺ T cells and B220⁺ T cells using MACS (Miltenyi Biotec) beads according to the manufacturer's protocol. For histology, organs were fixed in 10% formalin. Four microns paraffin embedded sections were cut and stained with hematoxylin and eosin (H&E). Minimally, four sections at multiple depths were analyzed.

In Vitro T Cell Activation and Migration Assays

5×10⁵ naïve CD4⁺ T cells from 5C.C7Rag1^{-/-} mice were stimulated with indicated concentrations of MCC₈₈₋₁₀₃ (moth cytochrome c) pulsed CHO cells expressing MHC class II, I-E^{k}, with or without scFvCD28 (variable fragment of CD28 antibody expressed as a trans-membrane protein on CHO cell surface). FACS sorted naive (CD4⁺CD8⁻CD19⁻CD11c⁻CD25⁻CD62L⁺CD44^{lo}) CD4⁺ T cells from WT and Itk^{-/-} mice were stimulated with 10 μg/ml SACD28 antibody at 37° C. for 24 hours, with recombinant IL-4 (2 ng/ml, R&D Systems) in some cultures. DKO LN cells were stimulated with 2 μg/ml Ionomycin for 2 hours prior to trans-endothelial migration assay. Ctla4^{-/-} LN cells were pre-incubated with ITK inhibitor, 10n (10 μg/ml), for 2 hours prior to trans-endothelial migration assay. Intra-cellular cytokine staining was performed after stimulating cells with PMA (50 ng/ml) and Ionomycin (750 ng/ml) for 4 hours in the presence of Golgi Stop and Plug (BD Bioscience). For trans-endothelial migration assays, a monolayer of SVEC4-10 (ATCC-CRL-2181; characterized as lymphatic, HEV, or blood microvascular endothelium) endothelial cells was established on the underside of a trans-well insert such that the migrating lymphocytes would first contact the basal surface of the endothelium, recapitulating migration in vivo, as previously described (Ledgerwood et al., Nat Immunol. 9, 42-53, 2008). Briefly, 100 μl of 0.1% Gelatin was added to the underside of trans-well insert and incubated for 2 hours at 37° C. The gelatin was then discarded and 5×10⁴ SVEC4-10 cells in 50 _{I}A complete media were added to the inverted insert. The set-up was incubated for 72 hours at 37° C. Confluency of cells

### Antibody List:

| Antibody | Clone | Format | Citation |
|---|---|---|---|
| GP38 | eBio8.1.1 | eFlour660 | us.ebioscience.com/mouse-podoplanin-antibody-efluor-660-811.htm |
| CD31 | 390 | eFlour450 | us.ebioscience.com/mouse-cd31-antibody-efluor-450-390.htm |
| CD86 | PO3.1 | PE | us.ebioscience.com/mouse-cd86-antibody-pe-po31.htm |
| CD80 | 16-10A1 | PerCP-Cy5′5 | bdbiosciences.com/ptProduct.jsp?prodId=744039&key=560526&par |
| | | | am=search&mterms=true&from=dTable |
| CD69 | H1.2F3 | PE/PE-Cy7 | us.ebioscience.com/mouse-cd69-antibody-pe-h12f3.htm |
| CD25 | PC61 | PE-Cy7 | bdbiosciences.com/ptProduct.jsp?prodId=375423&key=552880&par |
| | | | am=search&mterms=true&from=dTable |
| CD3 | 145-2C11 | PerCp-Cy5.5 | us.ebioscience.com/mouse-cd3e-antibody-percp-cy55-145-2c11.htm |
| IL17a | ebio17B7 | PerCP-Cy5.5 | us.ebioscience.com/mouse-il-17a-antibody-percp-cy55-ebio17b7.htm |
| IFNg | XMG1.2 | PE-Cy7 | bdbiosciences.com/ptProduct.jsp?prodId=441271&key=557649&par |
| | | | am=search&mterms=true&from=dTable |
| TNFa | MP6-XT22 | PE-Cy7 | bdbiosciences.com/ptProduct.jsp?prodId=441237&key=557644&par |
| | | | am=search&mterms=true&from=dTable |
| IL-10 | JES6-16E3 | PE | bdbiosciences.com/ptProduct.jsp?prodId=6182&key=554467&para |
| | | | m=search&mterms=true&from=dTable |
| IL-2 | JES6-5H4 | PE | bdbiosciences.com/ptProduct.jsp?prodId=6048&key=554428&para |
| | | | m=search&mterms=true&from=dTable |
| IL-4 | 11B11 | APC | us.ebioscience.com/mouse-il-4-antibody-apc-11b11.htm |
| Caspase-8 | 1G12 | | enzolifesciences.com/ALX-804-447/caspase-8-mouse-mab-1g12/ |
| CD4 | RM4-5 | PerCP-eFlour670 | us.ebioscience.com/mouse-cd4-antibody-percp-efluor-710-rm4- |
| | | | 5.htm |
| Annexin V | | FITC/PE | bdbiosciences.com/ptProduct.jsp?prodId=12827&key=556419&para |
| | | | m=search&mterms=true&from=dTable |
| Thy1.2 | 53-2.1 | V500 | bdbiosciences.com/ptProduct.jsp?prodId=1278583&key=561616&pa |
| | | | ram=search&mterms=true&from=dTable |
| Ly5.1 | A20 | PE/PE-Cy7 | us.ebioscience.com/mouse-cd451-antibody-pe-cy7-a20.htm |
| CD8a | 53-6.7 | APC-Cy7/ | bdbiosciences.com/ptProduct.jsp?prodId=442936&key=557654&par |
| | | PacBlue | am=search&mterms=true&from=dTable |
| CD44 | IM7 | eFlour 450 | us.ebioscience.com/human-mouse-cd44-antibody-efluor-450- |
| | | | im7.htm |
| CD62L | MEL-14 | PE/FITC | us.ebioscience.com/mouse-cd621-l-selectin-antibody-pe-mel-14.htm |
| TCRb | H57-597 | APC-eFlour | us.ebioscience.com/mouse-tcr-beta-antibody-apc-efluor-780-h57- |
| | | 780 | 597.htm |
| NK1.1 | PK136 | PE-Cy7 | bdbiosciences.com/ptProduct.jsp?prodId=375407&key=552878&par |
| | | | am=search&mterms=true&from=dTable |
| CCR7 | 4B12 | PE/PE-Cy7 | us.ebioscience.com/mouse-cd197-ccr7-antibody-pe-4b12.htm |
| CCR5 | C34-3448 | PE | bdbiosciences.com/ptProduct.jsp?prodId=80599&key=CCR5&para |
| | | | m=search&mterms=true&from=dTable |
| CCR9 | eBioCW-1.2 | PE | us.ebioscience.com/mouse-cd199-ccr9-antibody-pe-ebiocw-12.htm |
| LFA-1a | M17/4 | FITC | us.ebioscience.com/mouse-cd11a-antibody-fitc-m17-4.htm |
| CD103 | 2E7 | APC | us.ebioscience.com/mouse-cd103-antibody-apc-2e7.htm |
| A4b7 | DATK32 | PE | us.ebioscience.com/mouse-lpam-1-antibody-pe-datk32.htm |
| CXCR3 | CXCR3-173 | PerCP- | us.ebioscience.com/mouse-cd183-cxcr3-antibody-percp-cy55-cxcr3- |
| | | Cy5′5/APC | 173.htm |
| FOXP3 | FJK-16a | APC | us.ebioscience.com/mouse-rat-foxp3-antibody-apc-fjk-16s.htm |
| Ki67 | B56 | FITC/PE | bdbiosciences.com/ptProduct.jsp?prodId=15725&key=556026&para |
| | | | m=search&mterms=true&from=dTable |

was determined by staining insert with the cytosolic dye, CFSE, and visualizing under a fluorescence microscope. Where indicated, monolayers were stimulated with 25 ng/ml of TNFα for 24 hours, after which MACS purified CD4⁺ T cells were added to the upper chamber. The frequency of migrated cells was determined after 4 hours of incubation at 37° C.

Short-Term Migration Assays

Cells from LNs and spleen of 3-4 weeks old DKO and Ctla4^{-/-}) mice (both sexes) were labeled with CFSE and eFluor670, respectively, mixed at different ratios and 30×10⁶ cells were intravenously injected into age-matched, non-irradiated Rag1^{-/-} mice. 6-8 hours after transfer, peripheral blood lymphocytes were collected via tail bleed. Mice were then euthanized and lymphocytes isolated from perfused lungs and liver by collagenase digestion, as well as from SLOs. Frequencies of CD4⁺ and CD8⁺ T cells (TCRβ⁺ or Thy1⁺) were determined by flow cytometry in each tissue.

Treg Cell Assays

To generate mixed bone marrow chimeras (BMC), BM cells were flushed from the femurs and tibias of donor mice and depleted of CD4⁺ and CD8⁺ T cells using magnetic Dynal beads (Invitrogen). 5×10⁶ BM cells were injected into lightly irradiated (300 rads) Rag1^{-/-} (Ly5.2⁺) mice via the tail vein. Ly5.1⁺ wt BM was used for control experiments to verify normal reconstitutions. Mice were bled periodically to determine reconstitution of the peripheral T cell pool. Mice were sacrificed and analyzed at >8 wks post transfer. For colitis assays, 2×10⁵ sorted CD4⁺CD25⁺ Treg cells were co-transferred with 5×10⁵ WT CD4⁺ Tconv cells into Rag1^{-/-} mice. Mice were weighed weekly and examined for signs of colitis and wasting. Histological examination of colons was performed 6 weeks after adoptive transfer for transmural inflammation and leukocyte infiltration.

Viral Infections

Age and sex-matched mice were infected with 5×10⁴ PFU of LCMV Armstrong intra-peritoneally. To measure relative viral load, mice were euthanized at d11 post-infection, spleens removed and halved prior to homogenizing in 1 mL of RPMI complete media. RNA was isolated using TRIzol reagent and reverse-transcribed according to manufacturer's protocol. Real-time quantitative PCR was performed on the glycoprotein (GP) of LCMV and 18sRNA as previously described (McCausland et al., J Virol Methods. 147, 167-176, 2008). To track T cell responses infected mice were analyzed at various times by flow cytometry using viral epitope-specific MHC Class I tetramers and intracellular cytokine production. For Influenza A virus, age and sex-matched mice were infected intra-nasally under light isoflurane anesthesia with 2,500 Egg Infectious Doses 50 (EID₅₀) of A/PR8 strain of influenza A virus in 50 μl of PBS. Mice were weighed every day and euthanized at d18 post-infection. Viral titres were determined at d7 and 14 post-infection by quantifying viral RNA as previously described(McKinstry et al., J Immunol. 182, 7353-7363, 2009).

Gene Expression Profiling

CD4⁺CD25^{neg} T cells from 3 weeks old male Ctla4^{-/-} or DKO mice were sorted in duplicates, RNA was extracted using TRIzol reagent and microarray analysis performed with the Affymetrix MoGene 1.0 ST array based on the ImmGen protocol (immgen.org/Protocols/Total RNA Extraction with Trizol.pdf). Data were analyzed with modules of the GenePattern genomic analysis platform of the Broad Institute (broadinstitute.org/ cancer/software/genepattern). Differences in gene expression were identified by the Multiplot module (coefficient of variation <0.5; P value ≦0.05, Student's t-test; expressed genes defined as those with mean expression value >120 in at least one sample, 95% confidence interval, based on Immgen.org data processing of the MoGene 1.0 ST arrays) and functional categorization was performed using the Functional Annotation Tool DAVID for GO annotations. Heat maps were generated by row (gene) based hierarchical clustering (pairwise complete linkage) of data using the HierarchicalClustering module. Data were log transformed and row centered (subtraction from the mean) and a relative color scale based map generated using the HierarchicalClusteringViewer module. Microarray data deposition to GEO is in progress.

Confocal Microscopy and Immunofluorescence

2×10⁶ MACS enriched CD4⁺ T cells were fixed in 4% para-formaldehyde and permeabilized in 0.15% saponin and stained for actin with phalloidin-488 (5 Units, Invitrogen). Images were captured using a Nikon Eclipse E 600 microscope and IPlab Spectrum software (Scanalytics, VA). At least 15 fields were photographed and no less than 100 cells per sample were analyzed for each experiment. For confocal microscopy, CD4⁺ T cells were photographed using a Leica confocal microscope.

Preparation of Lung Slices for Microscopy

The procedure was performed as described (Sanderson, Pulm Pharmacol Ther. 24, 452-465, 2011) with some modifications. Briefly, mice were intravenously injected with 50 μM of CMTMR Orange dye (Invitrogen). After 30 minutes, 20-50×10⁶ CFSE (Invitrogen) labeled LN cells were intravenously injected and mice euthanized 5 minutes later. The trachea was cannulated, and lungs inflated with 0.9-1.3m1 of 1.8% LMP agarose in sHBSS (HBSS+20mM HEPES, pH 7.4). The agarose was gelled and a lung lobe was cut into 200 μm thick serial sections. Lung slices were adhered to a glass bottom dish (In vitro Scientific) by serial additions of a thin film of 2% agarose and bathed in 2 ml of phenol-red free DMEM containing HEPES and 10% FBS. Microscopy was performed at 35° C.

Fluorescence Microscopy

Fluorescence imaging was performed using a custom-built 2-photon or confocal microscope using a 40× or 60× objective with numerical apertures of 1.35 or 1.42, respectively. Cells were excited at 820 nm for 2-photon microscopy, and at 488 nm and 543 nm for confocal microscopy. Simultaneous fluorescence images were collected by separating the emitted fluorescence light with a 540 nm dichroic mirror in conjunction with a red (590 nm) and green (510 nm) barrier filter (Semrock). No substantial differences in the data were observed between the 2 microscopes. To follow lymphocyte motility, a time-lapse 2D image sequence was acquired by averaging 16 images (480×800 pixels at 15 images/second) every 5 seconds using Video Savant software (IO Industries). Each average image had dimensions of 240 μm×288 μm with a pixel resolution of 0.5×0.36 μm using a 40× objective. Data was analyzed using both Video Savant and Image J (NIH) software. Various descriptors of lymphocyte motility (displacement, roundness) were calculated using ImageJ software.

Diabetes Induction and Calculation of Insulitis Index

5 weeks old female NOD mice were treated with ITK inhibitor as indicated and extent of insulitis was determined by histopathological analysis of pancreatic sections. For T cell transfer experiments, 1×10⁶ sorted CD4⁺CD25^{neg} conventional T cells from 4 weeks old female BDC2.5/NOD TCR Tg mice were i.v. injected into 4wks old female NOD/scid mice. Mice were considered diabetic if they had 3 consecutive blood glucose readings of >250mg/dL. Insulitis index was calculated by scoring the islets as Grade 0: No infiltration; Grade 1: Peri-insulitis only; Grade 2: <20% of islet mass infiltrated; Grade 3: 75% of islet mass infiltrated; Grade 4: <20% of islet mass remaining as determined by insulin immuno-histochemistry. Insulitis index was calculated using the formula: I=(0×N0)+(1×N1)+(2×N2)+(3×N3)+(4×N4)/(NO+N1+N2+N3+N4); where N0, N1, N2, N3, and N4 are the number of islets showing Grade 0, 1, 2, 3, and 4 pathology respectively.

ITK Inhibitor Characterization and Purification

10n: ¹H NMR (400 MHz, DMSO-d₆) δ13.14-13.00 (br.s., 1H), 8.24-8.11 (br.s., 1H), 7.92-7.79 (br.s., 1H), 7.63 (d, J=3.8 Hz, 1H), 7.52 (s, 1H), 7.49 (d, J=8.4 Hz, 1H), 7.22 (d, J=3.8 Hz, 1H), 7.20 (dd, J=8.4, 1.0 Hz, 1H), 5.03 (s, 1H), 4.14 (s, 2H), 3.91 (d, J=12.7 Hz, 1H); 3.66 (d, J=12.7 Hz, 1H), 2.20 (q, J=6.4 Hz, 1H), 1.23 (s, 6H), 0.95 (d, J=6.4 Hz, 3H), 0.86 (s, 9H). ¹³C NMR (100 MHz, DMSO-d6) 6 168.4, 152.2, 140.8, 140.0, 136.1, 130.5, 129.2, 128.7, 125.8, 122.7, 122.3, 115.2, 111.0, 110.7, 70.8, 60.0, 52.5, 51.4, 34.0, 27.5, 26.2, 14.0; LC/MS: retention time 3.930 min (Gradient: 4% to 100% acetonitrile (0.05% TFA) over 7 min); HRMS: m/z (M+H⁺)=495.2538 (Calculated for C₂₆H₃₅N₆O₂S=495.2542).

Statistical Analysis

Sample size for in vivo studies including inhibitor treatment and diabetes induction were estimated by conducting pilot experiments. No samples or animals were excluded from the analyses. For animal studies, no randomization and blinding were used. Data were analyzed using Prism statistical software. Normally and non-normally distributed data were analyzed for significance by Student's t-test and Mann-Whitney test respectively. The variance was similar between groups being compared. Standard error and p-values are shown on individual graphs.

### Results

CD28-B7 Signals are Necessary for Autoreactive Ctla4^{-/-} T Cell Migration to Tissues

Pathogenic Ctla4^{-/-} CD4⁺ T cells recognize tissue self-antigens and represent a model of multi-organ autoimmunity mediated by self-reactive T cells. Cd28^{-/-}Ctla4^{-/-} mice are protected from lethal autoimmunity since self-reactive T cells cannot be activated (Mandelbrot et al., J Clin Invest. 107, 881-887, 2001). To determine whether B7-CD28 signals are also required for the migration of activated self-reactive T cells to tissues in vivo, we transferred activated Ctla4^{-/-} lymph node (LN) T cells into Cd80^{-/-} Cd86^{-/-}(B7^{-/-}) Rag1^{-/-} mice. In B7 sufficient Rag1^{-/-} mice, activated Ctla4^{-/-} T cells instigated an aggressive wasting disease and invaded most non-lymphoid tissues similar to intact Ctla4^{-/-} mice within three weeks (FIG. 1A). In the absence of B7, donor T cells were confined to secondary lymphoid organs (SLOs) and non-lymphoid tissues were mostly protected from infiltration by activated self-reactive T cells (FIG. 1A) and the recipients remained healthy for greater than 10 weeks post-transfer. The transfer of Ctla4^{-/-} T cells into MHC Class II-deficient Rag1^{-/-} mice, however, resulted in an intermediate disease course with 75% of mice displaying visible signs of autoimmunity and tissue infiltrates at 6 weeks post-transfer (FIG. 6A). These results suggested a more stringent requirement for B7-CD28 than MHC class II-TCR signals for activated Ctla4^{-/-} T cell trafficking into tissues.

While it is possible that the migratory potential of T cells might be programmed by B7 during their activation in SLOs, this alone cannot account for the aberrant migration of activated Ctla4^{-/-} T cells in B7-deficient hosts, given that the T cells were exposed to B7 prior to the transfer. It was therefore likely that functional B7 exists within non-lymphoid tissues. In the LNs, most stromal cell subsets, including endothelial cells (ECs), express some B7 (Fletcher et al., J Exp Med. 207, 689-697, 2010). ECs have also been previously shown to express costimulatory ligands and MHC class II molecules that activate T cells in a B7-dependent manner (Lozanoska-Ochser et al., J Immunol. 181, 6109-6116, 2008; Perez et al., Cell Immunol. 189, 31-40, 1998; Kreisel et al., J Immunol. 169, 6154-6161, 2002). We determined the expression of CD80 and CD86 on various stromal cell compartments in the lungs (FIG. 6B). While there was moderate amount of CD86 expression on CD45⁺hematopoietic cell subsets, CD45^{neg} stromal cell subsets expressed low but significant amounts of CD86 (FIG. 6C). Imaging studies also identified a population of CX3CR1⁺ immature DCs present on vessel walls in the lungs that project dendrites into the vessel lumen (Thornton et al., J Exp Med. 209, 1183-1199, 2012). These results suggested that low amounts of B7 are available in tissues for blood-borne T cells. To determine whether Ctla4^{-/-} T cells can interact with ECs in a CD28-dependent manner, we tested the ability of activated Ctla4^{-/-} T cells to migrate across B7 expressing SVEC4-10 cells (Ledgerwood et al., Nat Immunol. 9, 42-53, 2008; Kreisel et al., J Immunol. 169, 6154-6161, 2002; Thornton et al., J Exp Med. 209, 1183-1199, 2012; Ledgerwood et al., Nat Immunol. 9, 42-53, 2008). While Ctla4^{-/-} T cells trafficked efficiently across a TNFa stimulated EC barrier, blockade of B7-CD28 interactions by neutralizing B7 antibodies significantly curtailed the migration of activated Ctla4^{-/-} CD4⁺ T cells (FIG. 6D).

Altered Migratory Behavior of Ctla4^{-/-} T Cells in B7-Deficient Hosts

To characterize autoreactive T cell motility in tissues, we performed 2-photon imaging of fluorescently labeled Ctla4^{-/-} T cells in lung vasculature of WT and B7-deficient mice (Cahalan et al., Ann Rev Immunol. 26, 585-626, 2008; Sanderson, Pulm Pharmacol Ther. 24, 452-465, 2011). Ctla4^{-/-} T cells transferred into WT mice were highly motile within blood vessels in lung slices, made frequent stable contacts with and often migrated across vessel walls (FIG. 1B). They showed significant mean displacement within the time frame of recording (FIG. 1C, 1D) and had the characteristic elongated morphology of migrating cells (FIG. 1B, 1E). Contrary to their behavior in blood vessels of B7-sufficient mice, Ctla4^{-/-} T cells did not make stable contact with endothelial cells, lost directionality and assumed a distinct circular morphology in ex vivo B7-deficient lung tissues (FIG. 1B-1E). These results indicated that CD28-B7 interaction is required for Ctla4^{-/-} T cell trafficking in tissues.

ITK Deficiency Prevents Lethality but not Lymphoproliferation in Ctla4^{-/-} Mice

To identify a CD28 signaling molecule required for T cell trafficking, we focused on ITK that is activated synergistically by both the TCR and CD28. To show that ITK can function downstream of CD28, we stimulated naïve WT and Itk^{-/-} CD4⁺ T cells with a CD28 super-agonist (SACD28) antibody (Dennehy et al., J Immunol. 176, 5725-5729, 2006) that can trigger T cell activation without overt TCR signaling. WT naïve T cells responded to SACD28 crosslinking by up-regulating activation markers CD25 and CD69, but Itk^{-/-} CD4⁺ T cells failed to do so (FIG. 6E).

CTLA-4 counterbalances CD28 signals and its absence in mice results in the robust activation of tissue antigen specific autoreactive T cells. To test if ITK has specific function in self-reactive T cell trafficking, we generated Itk^{-/-}Ctla4^{-/-} double knockout (DKO) mice. Unlike Ctla4^{-/-} mice that die by 3-4 weeks of age of rampant multi-organ autoimmunity, DKO mice were healthy and had a significantly extended life span (FIG. 2A). Yet, DKO mice had engorged peripheral LNs (FIG. 2B), 20 times larger than those of normal mice, packed with proliferating (Ki67⁺), activated) (CD44^{hi}CD62L^{lo}) T cells (FIG. 2B, 2C) that were fully capable of effector cytokine production (FIG. 7A) and exhibiting a similar degree of apoptosis as Ctla4^{-/-} T cells (FIG. 7B). These results indicated that ITK signaling was not required for the activation and expansion of autoreactive Ctla4^{-/-} T cells. Consistent with this conclusion, naive 5C.C7TCR Tg CD4⁺ T cells (CD44^{lo}CD62L^{hi}) from 5C.C7TgItk^{-/-}Ctla4^{-/'}Rag1^{-/-} mice responded normally to stimulation with the cognate moth cytochrome c (MCC) peptide (FIG. 7C). Lastly, global gene expression profiling of CD4⁺T cells from Ctla4^{-/-} and DKO mice showed that these two populations were molecularly convergent, with less than 0.5% of genes differentially expressed (FIG. 7D), supporting the conclusion that the activation and functional states of Ctla4^{-/-} T cells were not grossly altered by the loss of ITK.

Lack of Autoimmune Tissue Pathology in Itk^{-/-}Ctla4^{-/-} DKO Mice

Similar to Ctla4^{-/-} mice, DKO mice exhibited an increased frequency of FOXP3⁺ Treg cells in peripheral SLOs (FIG. 2C). However, the lack of CTLA-4 rendered these Treg cells functionally impaired, as they were unable to regulate colitogenic naïve WT T cells in vivo (FIG. 7E). Further, in the presence of WT Treg cells in mixed bone marrow chimeras, DKO T cells were prevented from activation (FIG. 7F), showing that the ITK-independent rampant T cell expansion in DKO mice was caused by the lack of CTLA-4 in Treg cells (Friedline et al., J Exp Med. 206, 421-434, 2009).

Despite defective Treg cells and extreme lymphoproliferation, DKO mice did not exhibit autoimmune tissue infiltration histologically (FIG. 3A). The few T cells present in tissues of older DKO mice did not exhibit differences in cell survival (FIG. 7B). The compromised tissue infiltration by activated DKO T cells was a cell-intrinsic defect, as reconstitution of Tcrb^{-/-} mice with DKO T cells did not cause deterioration in health, whereas transferred Ctla4^{-/-} T cells caused severe autoimmune pathology (FIG. 3B) and death of mice by 4-6 weeks after transfer. Pertussis toxin (PTx) facilitates the movement of autoreactive T cells to brain tissues to induce EAE in mice (Goverman et al., Crit Rev Immunol. 17, 469-480, 1997). Treatment of DKO mice with PTx led to a rapid wasting disease with death starting at one month post-treatment. Importantly, there were increased lymphocytic infiltrates into organs accompanied by robust effector cytokine production by CD4⁺ T cells (FIG. 3C).

Critically, DKO mice could mount effective anti-viral T cell responses and clear infections with the A/PR8 strain of Influenza A and LCMV Armstrong viruses similar to WT and Itk^{-/-} mice (FIG. 7G, 7H). There was no significant difference in the composition of lymphocyte subsets activated by infections or the extent of lymphocyte infiltration into tissues by infected DKO mice compared to control WT and Itk^{-/-} mice. These results indicated that DKO T cells are not inert and irreversibly excluded from tissues, and that ITK appears to only license aberrantly activated, self-reactive T cells to accumulate in tissues.

Chemokine Responses of Itk^{-/-}Ctla4^{-/-} DKO T Cells

We next determined if the chemokine dependent response of Ctla4^{-/-} T cells was altered because of ITK deficiency. Global gene expression profiling and confirmatory flow cytometry assays revealed no significant differences in the expression of various adhesion molecules and most chemokine receptors in Ctla4^{-/-} and DKO T cells (FIG. 8A, 8B). Semi-quantitative RT-PCR analysis showed similar levels of expression of the SIP receptor, important for LN exit, and comparable migration to SP ligand by Ctla4^{-/-} and DKO T cells (FIG. 8C). Increased frequency and numbers of circulating activated CD4⁺ T cells in blood of DKO mice further indicated that DKO T cells can exit the LNs (FIG. 8D). An exception to this general pattern of similarity in chemokine receptor expression between Ctla4^{-/-} and DKO T cells was the decreased expression of CXCR3 (Liu et al., Curr Top Dev Biol. 68, 149-181, 2005) on activated DKO CD4⁺ T cells and their failure to migrate to CXCL-11 in vitro (FIG. 9A, 9B). However, while Cxcr3^{-/-}Ctla4^{-/-} mice displayed a small but significant extension in lifespan relative to Ctla4^{-/-} mice, massive lymphocytic infiltrates were still evident in most organs (FIG. 9C, 9D). Thus, reduced expression of CXCR3 on DKO CD4⁺ T cells likely contributes to, but cannot fully account for the constrained trafficking of DKO T cells into tissues.

Itk^{-/-}Ctla4^{-/-} DKO T Cells are Inefficient in Non-Lymphoid Tissue Entry

To determine whether DKO T cells are selectively impaired in migration to tissues, we performed a competitive short-term tissue homing assay and calculated the ratios of fluorescently labeled DKO and Ctla4^{-/-} T cells in lymphoid and non-lymphoid tissues 6-8 hours after intravenous injection into Rag1^{-/-} mice. At this early time point, the transferred cells remained undivided and there was no difference in the expression of apoptosis marker AnnexinV in all tissues (FIG. 9E). In the lungs and liver, the normalized ratios of DKO/Ctla4^{-/-} CD4⁺ and CD8⁺ T cells, the homing index (HI), was <1.0 (FIGS. 4A and 9E), indicating a significant advantage of Ctla4^{-/-} T cells in repopulating these tissues. Reciprocally, a HI of >1.5 in the LNs indicated an enhanced accumulation of DKO T cells in lymphoid tissues. The difference was observed regardless of initial input ratios ranging from 2:1 to 1:4 of Ctla4^{-/-}:DKO cells. These results indicate that in the short time frame of this assay, DKO T cells do not migrate to non-lymphoid tissues as efficiently as Ctla4^{-/-} T cells. This disparity is most likely an underestimate since at the time of T cell isolation for transfer, most T cells in DKO reside in lymphoid tissues, whereas Ctla4^{-/-} T cells are replete in most non-lymphoid tissues.

Itk^{-/-}Ctla4^{-/-} DKO T Cells are Defective in Trans-Endothelial Migration

Morphological changes induced by cytoskeletal reorganization enable activated T cells to traffic across endothelial cells and exit the blood circulation to enter tissues (Burkhardt et al., Ann Rev Immunol. 26, 233-259, 2008). Activated DKO CD4⁺ T cells had impaired F-actin polarization relative to activated Ctla4^{-/-} T cells when examined directly ex vivo (FIGS. 4B and 8E). Further, unlike Ctla4^{-/-} T cells, a significantly lower frequency of activated DKO CD4⁺FOXP3^{neg} T cells was able to migrate across an endothelial cell layer in vitro (FIG. 4C). Pre-activation of DKO T cells with Ionomycin, a Ca²⁺ ionophore that can partly substitute for CD28 signaling, increased the frequency of DKO T cells that were able to migrate across the endothelium, supporting a CD28 signaling defect in DKO cells (FIG. 8F). Similarly, migration of Ctla4^{-/-} T cells across endothelial cells in vitro was blocked by a small molecule ITK inhibitor (Readinger et al., Proc Natl Acad Sci USA 105, 6684-6689, 2008) (FIG. 8G). 2-photon imaging of DKO lymphocytes in lung slices showed that, in contrast to Ctla4^{-/-} T cells, DKO T cells exhibited a random movement within blood vessels in lung tissue and were morphologically distinct (FIGS. 4D-G). The migratory properties of DKO cells were strikingly similar to that of Ctla4^{-/-} T cells in B7-deficient lung slices, suggesting that an impairment of CD28 signaling in DKO T cells was responsible for their altered trafficking.

Small Molecule ITK Inhibitors Regulate Autoimmunity In Vivo

Data so far raised the possibility that pharmacological inhibition of ITK might moderate autoimmune disease pathogenesis. We tested this using two validated inhibitors of ITK, BMS509744 (Lin et al., Biochem. 43, 11056-11062, 2004) and 10n (Riether et al., Bioorg Med Chem Lett. 19, 1588-1591, 2009). Treatment of Ctla4^{-/-} mice with ITK inhibitors significantly increased their lifespan (FIG. 5A). The pharmacological inhibition of ITK did not alter the activation state or proliferative capacity of Ctla4^{-/-} T cells (FIG. 10A), but led to a significant increase in the size of LNs (FIG. 10B), consistent with the earlier genetic studies (FIGS. 2B, 2C). However, despite the increased longevity of the mice, lymphocytic infiltration was observed in most tissues. This result was not unexpected given the relatively poor pharmacokinetics of the ITK inhibitors, combined with the rapid-onset, extremely destructive disease occurring in Ctla4^{-/-} mice.

We next tested the ability of ITK inhibitors to prevent β-islet infiltration in non-obese diabetic (NOD) mice in which self-reactive T cell precursor frequencies are relatively low at the onset of disease. Administration of 10n to female NOD mice caused an increase in the cellularity of the LNs, similar to that observed in the treated Ctla4^{-/-} mice (FIG. 10C). Importantly, 10n treatment reduced the migration of self- reactive T cells into pancreatic β-islets of NOD mice by 50% (FIGS. 5B, 5C). Given the prohibitive amount of 10n required to perform long-term studies of the efficacy of the ITK inhibitor to prevent fulminant diabetes in NOD mice, we instead chose to examine whether 10n could block diabetogenic BDC2.5/NOD CD4⁺ T cells from causing islet cell destruction. Transfer of BDC2.5/NOD CD4⁺CD25^{neg} T cells into young NODI Scid mice (Peterson et al., Diabetes 45, 328-336, 1996), with parallel 10n treatment moderated insulitis (FIG. 5D) and diminished the onset of diabetes (FIG. 5E).

CTLA-4 and CD28 are critical for regulating autoimmunity such as T1D in mice and humans, but are often dispensable for responses against foreign pathogens (Lenschow et al., Immunity 5, 285-293, 1996; Friedline et al., J Exp Med. 206, 421-434, 2009; Bachmann et al., J Immunol. 160, 95-100, 1998; Shahinian et al., Science 261, 521-652, 1993). Most in vivo studies support the role of ligand competition as the primary mode of CTLA-4 inhibition of CD28 signals (Masteller et al., J Immunol. 164, 5319-5327, 2000; Collins et al., Immunity 17, 201-210, 2002; Yokosuka et al., Immunity 33, 326-339, 2010; Puccetti et al., Nat Rev Immunol. 7, 817-823, 2007; Qureshi et al., Science 332, 600-603, 2011). We show here that CD28-B7 interactions and ITK regulate the trafficking of self-reactive T cells to tissues. Given the established biochemical connection between CD28 and ITK, these data support the model that one Tconv cell-intrinsic function of CTLA-4 is to modulate the CD28-ITK pathway controlling self-reactive T cell motility in tissues.

CD28 signaling is necessary for the activation of self-reactive Ctla4^{-/-} T cells in vivo (Mandelbrot et al., J Clin Invest. 107, 881-887, 2001). While altering specific downstream mediators of CD28 in Ctla4^{-/-} mice was predicted to reveal pathways that contribute to the pathogenesis of T cell mediated autoimmunity, the challenge was to identify components that have biased function in specific facets of CD28-mediated disease pathogenesis. ITK was an ideal candidate, since the loss-of-function of ITK does not completely abolish TCR-CD28 signaling, but primarily impairs the PI3K-PLCγ1 pathway (Berg et al., Ann Rev Immunol. 23, 549-600, 2005). Consequently, naïve DKO

CD4⁺T cells respond to their cognate antigens comparably to WT T cells, and DKO mice are replete with activated T cells; these findings established that the CD28 costimulatory signals necessary for the initial activation of Ctla4^{-/-} T cells are intact in the absence of ITK. Further, DKO mice are effective in pathogen clearance, again confirming that T cell activation and differentiation are not globally impaired. Hence, the inability of activated DKO T cells to enter tissues and to cause fatal autoimmunity revealed a specific function of ITK in self-reactive T cell trafficking. In addition to CD28 signaling, ITK has been implicated in integrin and chemokine receptor signaling (Woods et al., EMBO J. 20, 1232-1244, 2001; Takesono et al., Curr Biol. 14, 917-922, 2004; Sahu et al., J Immunol. 180, 3833-3838, 2008); however, the major consequence of this in Itk^{-/-} mice is impaired lymphocyte trafficking to the LNs. In models of infectious diseases, ITK-deficient T cells did not show significant alterations in their migratory property (Bachmann et al., J Virol. 71, 7253-7257, 1997; Au-Yeung et al., J Immunol. 176, 3895-3899, 2006). DKO T cells were impaired in responding to CXCR3 ligands in vitro; however despite this, Cxcr3^{-/-}Ctla4^{-/-} mice had extensive T cell infiltrates into tissues and a short life span, indicating that possible defects in CXCR3 signaling due to ITK-deficiency cannot fully account for the lack of T cell infiltrates in DKO mice.

Lastly, we showed that a small molecule ITK inhibitor prevents the migration of activated diabetogenic BDC2.5 CD4⁺ T cells into the pancreas. These findings indicate that the requirement for ITK in autoimmune pathogenesis first identified with Ctla4^{-/-} T cells is not unique and is likely to be relevant for self-reactive T cells in various human organ specific autoimmune disorders. Costimulatory blockade is a major therapeutic strategy for autoimmune diseases such as rheumatoid arthritis and T1D. Abatacept, a CD28 antagonist, has achieved moderate success in T1D patients, leading to the preservation of beta-cell mass for over two years (Orban et al., Lancet 378, 412-419, 2011). However, this approach ultimately fails due to the increased antigen sensitivity of autoreactive T cells, and their relative independence from costimulation for their activation. Another major drawback is the failure to specifically target auto-reactive T cells as B7 blockade can interfere with Treg cell function (Salomon et al., Immunity 12, 431-440, 2000). In contrast, focused approaches aimed at regulating self-reactive T cell migration to organs to limit immune pathology are beneficial in treating severe autoimmunity (Bauer et al., Proc Natl Acad Sci USA 106, 1920-1925, 2009). ITK has been shown to have a similar functional repertoire in human T cells as in mice, including the ability to modulate T cell actin polarization (Readinger et al., Proc Natl Acad Sci USA 105, 6684-6689, 2008; Dombroski et al., J Immunol. 174, 1385-1392, 2005; Guo et al., Mol Pharmacol. 82, 938-947, 2012). The data indicate that ITK inhibitors can become an alternate strategy to treat diverse human T cell mediated organ-specific autoimmune diseases, while allowing pathogen-driven immune responses to occur.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treating a subject with an organ-specific T cell mediated autoimmune disease, the method comprising:
identifying a subject in need of treatment for an organ-specific T cell mediated autoimmune disease; and
administering to the subject a therapeutically effective amount of an interleukin-2-inducible T cell kinase (ITK) inhibitor,
thereby treating the subject with the organ-specific T cell mediated autoimmune disease.

2. The method of claim 1, wherein the ITK inhibitor is selected from the group consisting of BM S509744, ibrutinib, 10n, 2-amino -5 -(thio aryl)thiazo le , 5-aminomethylbenzimidazole, 2-amino-5-[(thiomethyparyl]thiazole, and biaryl thiophene.

3. The method of claim 1, wherein the method further comprises administering abatacept, secukinumab, or infliximab to the subject.

4. The method of claim 1, wherein the organ-specific T cell mediated autoimmune disease is selected from the group consisting of Type 1 diabetes, Hashimoto's thyroiditis, multiple sclerosis, non-infectious uveitis, Sjögren's syndrome, primary biliary cirrhosis, autoimmune hepatitis, and ankylosing spondylitis.

5. The method of claim 1, wherein the method comprises administering the ITK inhibitor to the subject orally, intravenously, or by injection.

6. The method of claim 1, wherein the subject is a mammal.

7. The method of claim 1, wherein the subject is a human.

8. The method of claim 1, wherein the method inhibits migration of autoreactive T cells into a target tissue.

9. The method of claim 1, wherein migration of alloreactive T cells is unaffected.

10. An interleukin-2-inducible T cell kinase (ITK) inhibitor for use in treating an organ-specific T cell mediated autoimmune disease in a subject.

11. The ITK inhibitor of claim 10, which is selected from the group consisting of BMS509744, ibrutinib, 10n, 2- amino -5 -(thio aryl)thiazo le, 5 -amino methylbenzimidazole, 2-amino-5-[(thiomethyparyl]thiazole, and biaryl thiophene.

12. The ITK inhibitor of claim 10, in combination with abatacept, secukinumab, or infliximab, for use in treating an organ-specific T cell mediated autoimmune disease.

13. The ITK inhibitor of claim 10, wherein the organ-specific T cell mediated autoimmune disease is selected from the group consisting of Type 1 diabetes, Hashimoto's thyroiditis, multiple sclerosis, non-infectious uveitis, Sjögren's syndrome, primary biliary cirrhosis, autoimmune hepatitis, and ankylosing spondylitis.

14. The ITK inhibitor of claim 10, which is formulated to be administered to the subject orally, intravenously, or by injection.

15. The ITK inhibitor of claim 10, wherein the subject is a mammal.

16. The ITK inhibitor of claim 10, wherein the subject is a human.

17. The ITK inhibitor of claim 10, which inhibits migration of autoreactive T cells into a target tissue.

18. The ITK inhibitor of claim 10, wherein migration of alloreactive T cells is unaffected.
